SEQUENCE LISTING (2 sequences)

SEQ ID NO: 1 - DNA, 481 bp, Escherichia coli
  CDS (239)..(361)
tgcctctcattgaagtatgatggctatttgacactatcctttacccacgctcaacagtttaataacctgccagcaataagggatgttgtttaacttaagtcaaaaaaatagcgaattttccaacgaacaaaagctaaatatcgcaaaaacctcagtaaaaatcttgctggagctattattgctaagtaacatttaccccctgaagttaatggatcaatcaagagagatgtgggctgtaatgaatcgtcttattgaattaacaggttggatcgttcttgtcgtttcagtcattcttcttggcgtggcgagtcacattgacaactatcagccacctgaacagagtgcttcggtacaacacaagtaagctctgcacttgtggagcgacatgctgcccgtccgggtgcatgttttcacttgtcggatattaaaccaggaatttattatcttgttcgatgttgttggtgattgtcagggatagtaa

SEQ ID NO: 2 - protein, 41 aa, Escherichia coli
MNRLIELTGWIVLVVSVILLGVASHIDNYQPPEQSASVQHK

(19) United States
(12) Patent Application Publication
(10) Pub. No.: US 2002/0051982 A1
(43) Pub. Date: May 2, 2002

(54) Novel BLR molecules affecting antibiotic susceptibility

(76) Inventors: Stuart B. Levy, Boston, MA (US); Rebecca S.Y. Wong, Port Coquitlam (CA); Laura M. McMurry, Cambridge, MA (US)

Correspondence Address: LAHIVE & COCKFIELD, 28 STATE STREET, BOSTON, MA, 02109, US

(21) Appl. No.: 09/828,456

(22) Filed: Apr. 6, 2001

Related U.S. Application Data
(60) Provisional application No. 60/195,505, filed on Apr. 6, 2000.
(60) Provisional application No. 60/218,380, filed on Jul. 14, 2000.

(51) Int. Cl. C12Q 1/68; G01N 33/554; G01N 33/569; C07H 21/04; C12N 9/00; C12N 1/21

(52) U.S. Cl. 435/6; 435/7.32; 435/183; 435/252.3; 536/23.2

(57) ABSTRACT

A novel 358 base pair sequence encoding a membrane protein that affects susceptibility to antibiotics that affect peptidoglycan synthesis in microbes is described. BLR nucleic acid and polypeptide molecules are provided. In addition, screening assays to identify agents that modulate BLR activity are described.

### RELATED APPLICATIONS

This application claims priority to U.S. provisional patent application Ser. No. and 60/195,505 filed Apr. 6, 2000, and to U.S. provisional patent application Ser. No. 60/218,380, filed on Jul. 14, 2000, the entire contents of both of these applications are hereby incorporated by reference.

### BACKGROUND OF THE INVENTION

Molecules that promote microbial resistance to antibiotics can be either extrachromosomally or chromosomally specified. Many nucleotide sequences that modulate antibiotic resistance remain to be identified. Despite the fact that the E. coli genome project has been completed and the sequence is now available in the public domain (Blattner et al. 1997 Science. 277:1453), nearly 40% of E. coli's 4288 actual and proposed open reading frames (ORFs) are completely uncharacterized. In addition to ongoing efforts to decipher the coding regions, efforts have also been made to characterize the intergenic regions. Some of these regions contain large repetitive sequences, some contain ORFs encoding proteins of small size, and some contain putative gene regulatory regions. Still, some of these greater than 600 bp intergenic regions have not been assigned any regulatory or coding function. The identification of additional regions of bacterial genomes that affect susceptibility to antibiotics will be of great benefit in controlling antibiotic resistance.

### SUMMARY

The present invention represents an important advance in the battle against drug resistance by demonstrating a 358 base pair sequence encoding a novel membrane protein that affects susceptibility to antibiotics that inhibit peptidoglycan synthesis in microbes. A 6.5 kb BamHI chromosomal fragment from RW583 containing the phoA and kan genes from TnphoA was identified by cloning into the BamHI site of pBR322 and selection in medium comprising kanamycin. The sequence of the clone revealed that the insertion was at nucleotide 1702674 of the genome (min 36.6) in a hypothetical intergenic region of 602 base pairs between two divergent ORFs (b1624 (ORF359, putative oxidoreductase, on the—strand) and b1625 (ORF71, putative histone-like negative regulator)). Examination of the antibiotic resistance profile of this mutant showed that the mutant was more susceptible to a wide spectrum of antibiotics that affect peptidoglycan synthesis than the parental strain. Complementation of the mutant with a 358 base pair sequence restored the antibiotic susceptibility phenotype of the parent strain; the 358 base pair sequence was found to specify a protein.

In one aspect, the invention provides an isolated nucleic acid molecule comprising the nucleotide sequence set forth in SEQ ID NO:1.

In another aspect, the invention provides an isolated nucleic acid molecule encoding a polypeptide comprising the amino acid sequence set forth in SEQ ID NO: 2.

In still another aspect, the invention provides an isolated nucleic acid molecule comprising a nucleotide sequence which is at least 50% identical to the nucleotide sequence of SEQ ID NO:1 or a complement thereof selected from the group consisting of;

a) a nucleic acid molecule comprising a fragment of at least 100 nucleotides of a nucleic acid molecule comprising the nucleotide sequence of SEQ ID NO:1 complement thereof;

b) a nucleic acid molecule which encodes a polypeptide comprising an amino acid sequence at least about 50% homologous to the amino acid sequence of SEQ ID NO:2; and

c) a nucleic acid molecule which encodes a fragment of a polypeptide comprising the amino acid sequence of SEQ ID NO:2, wherein the fragment comprises at least 15 contiguous amino acid residues of the amino acid sequence of SEQ ID NO:2.

In yet another aspect, the invention provides an isolated nucleic acid molecule which hybridizes to the nucleic acid molecule of any one of claims 1, 2, or 3 under stringent conditions.

In another aspect, the invention provides an isolated nucleic acid molecule comprising a nucleotide sequence which is complementary to the nucleotide sequence of the nucleic acid molecule of any one of claims 1, 2, or 3.

In yet another aspect, the invention provides an isolated nucleic acid molecule comprising the nucleic acid molecule of any one of claims 1, 2, or 3, and a nucleotide sequence encoding a heterologous polypeptide.

In still another aspect, the invention is directed to a vector comprising the nucleic acid molecule of any one of claims 1, 2, or 3.

In one embodiment, the vector is an expression vector.

In another embodiment, a host cell is transfected with an expression vector.

In one aspect, the invention provides a method of producing a polypeptide comprising culturing the host cell of claim 9 in an appropriate culture medium to, thereby, produce the polypeptide.

In another aspect, the invention provides an isolated polypeptide selected from the group consisting of:

a) a fragment of a polypeptide comprising the amino acid sequence of SEQ ID NO:2, wherein the fragment comprises at least 15 contiguous amino acids of SEQ ID NO:2;

b) a naturally occurring homolog of a BLR polypeptide comprising the amino acid sequence of SEQ ID NO:2, wherein the naturally occurring homolog is isolated from a pathogenic bacterium and is encoded by a nucleic acid molecule which hybridizes to a nucleic acid molecule consisting of SEQ ID NO:1;

c) a polypeptide which is encoded by a nucleic acid molecule comprising a nucleotide sequence which is at least 50% identical to a nucleic acid molecule comprising the nucleotide sequence of SEQ ID NO:1;

d) a polypeptide comprising an amino acid sequence which is at least 50% identical to the amino acid sequence of SEQ ID NO:2.

In one embodiment, the invention provides a polypeptide comprising the amino acid sequence of SEQ ID NO:2.

In another aspect, the invention provides an antibody which selectively binds to a BLR polypeptide.

In another aspect, the invention provides an agonist of a BLR polypeptide.

In still another aspect, the invention provides an antagonist of a BLR polypeptide.

In another aspect, the invention provides a method for identifying compounds that modulate antibiotic resistance in a microbe comprising:

contacting a BLR polypeptide with a test compound;

determining the ability of the test compound to modulate the activity or expression of a BLR polypeptide; and

selecting those compounds that modulate the activity of the BLR polypeptide to thereby identify compounds that modulate antibiotic resistance.

In one embodiment, a BLR polypeptide is present in a microbial cell.

In one embodiment, a BLR polypeptide is heterologous to the cell in which it is present.

In one embodiment, the microbial cell is an E. coli cell.

In one embodiment, the step of determining comprises measuring the effect of the test compound on the ability of the microbial cell to grow in the presence of an antibiotic.

In one embodiment, the antibiotic is an antibiotic that affects peptidoglycan synthesis selected from the group consisting of a beta lactam, cycloserine, and bacitracin.

In one embodiment, the step of determining comprises measuring the efflux of the test compound or a marker compound from the cell.

In one embodiment, the BLR polypeptide is contacted with the test compound in vitro.

In another aspect, the invention provides a method for identifying compounds that modulate antibiotic resistance in a microbe comprising:

contacting an isolated BLR nucleic acid molecule with a test compound;

determining the ability of the test compound to bind to the isolated BLR nucleic acid molecule; and

selecting those compounds that bind to the BLR nucleic acid molecule to thereby identify compounds that modulate antibiotic resistance.

In one embodiment, the BLR nucleic acid molecule comprises the nucleotide sequence shown in SEQ ID NO:1.

In another aspect, the invention provides a method for identifying a protein that interacts with a BLR nucleotide sequence, comprising:

contacting a BLR nucleotide sequence with a microbial extract under conditions which allow interaction of components of the extract with the BLR nucleotide sequence; and measuring the ability of the BLR nucleotide sequence to interact with the components thereby identify a protein that binds to a BLR nucleotide sequence.

In another embodiment, the invention provides a method for identifying a compound that modulates the ability of a BLR nucleic acid molecule to interact with a BLR binding polypeptide, comprising:

contacting at least one of a BLR nucleotide sequence and a BLR binding polypeptide with a test compound under conditions which allow interaction of the compound with at least one of the BLR nucleotide sequence and the BLR binding polypeptide; and measuring the ability of the compound to modulate the interaction of the BLR nucleotide sequence with the BLR binding polypeptide to thereby identify a compound that modulates the ability of a BLR nucleotide sequence to interact with a BLR binding polypeptide.

In yet another aspect, the invention provides a method for identifying a compound that modulates the ability of a BLR polypeptide to interact with a BLR binding polypeptide, comprising:

contacting at least one of a BLR polypeptide and a BLR binding polypeptide with a test compound under conditions which allow interaction of the compound with at least one of the BLR polypeptide and the BLR binding polypeptide; and measuring the ability of the compound to modulate the interaction of the BLR polypeptide with the BLR binding polypeptide to thereby identify a compound that modulates the ability of a BLR polypeptide to interact with a BLR binding polypeptide.

### BRIEF DESCRIPTION OF THE DRAWINGS

FIG. 1 shows the DNA sequence surrounding the TnphoA insertion site (marked with the vertical arrow). The insertion site follows a C residue. The amino acid sequence of BLR is given; its ATG translational start is in large, bold font.

### DETAILED DESCRIPTION

A mini-TnphoA insertion in a 602 base pair “intergenic” region of the Escherichia coli chromosome at genomic nucleotide 1702674 gave rise to a membrane-bound PhoA fusion protein and a 2 to 4 fold increase in the intrinsic susceptibility to a wide spectrum of antibiotics that inhibit peptidoglycan synthesis without changing beta-lactamase activity. A clone bearing only 358 base pairs of the beta lactam resistance “BLR” region restored beta-lactam resistance to the parental level. Two amber mutations in the clone prevented this restoration and were counteracted by an amber suppressor, proving that the active species is a protein. The BLR protein has 41 amino acids, with a single predicted transmembrane helix but no clear homology to any other protein. There is a transcriptional start 39 base pairs upstream from the translational start.

The novel membrane protein encoded by the 358 bp nucleotide sequence located in an approximately 600 bp intergenic region of the E. coli genome promotes susceptibility to a wide spectrum of antibiotics that inhibit peptidoglycan synthesis. Accordingly, the invention provides methods and compositions for controlling resistance to antibiotic and non antibiotic compounds.

Before further description of the invention, certain terms employed in the specification, examples and appended claims are, for convenience, collected here.

I. Definitions

The language “Beta Lactam-358 (BLR)” nucleic acid molecules as used herein includes nucleic acid molecules having a nucleotide sequence related to the BLR nucleotide sequence shown in SEQ ID NO:1 or to the complement thereof and/or encode polypeptides which share certain functional features with the BLR polypeptide sequence of SEQ ID NO:2. For example, BLR nucleotide sequences share nucleotide sequence similarity (e.g., identity) with the BLR nucleotide sequence shown in SEQ ID NO:1 and encode “BLR polypeptides,” i.e., polypeptides which share amino acid sequence identity and which share a BLR polypeptide activity with the BLR amino acid sequence shown in SEQ ID NO:2. BLR polypeptides preferably comprise less than about 75 to about 50 amino acid residues. Exemplary BLR polypeptides include BLR homologues of approximately 41 residues in the incomplete genomic sequence of Salmonella typhimurium (85% identity), S. typhi (82% identity), and S. paratyphi A (82% identity), and a homologue of 45 residues in Klebsiella pneumoniae (49% identity). Preferably BLR polypeptides are membrane proteins.

BLR polypeptides share a BLR activity. The term “activity” with respect to a BLR polypeptide includes the ability of a BLR polypeptide to promote drug resistance in a cell in which it is expressed (e.g., by promoting drug efflux from a cell or by inhibiting lysis of the cell) and/or the ability of a BLR polypeptide to bind to molecules to which it normally binds. Preferably, BLR polypeptides can increase resistance to antibiotics that inhibit peptidoglycan synthesis when expressed in a cell. Preferably, BLR polypeptides do not posses P lactamase activity.

As used herein, the term “nucleic acid molecule(s)” includes polyribonucleotides or polydeoxribonucleotides, which may be unmodified RNA or DNA or modified RNA or DNA. As such, “nucleic acid molecule(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “nucleic acid molecule” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. As used herein, the term “nucleic acid molecule” also includes DNAs or RNAs as described above that contain one or more modified bases. Thus, DNAs or RNAs with backbones modified for stability or for other reasons are “nucleic acid molecule(s)” as that term is intended herein. Moreover, DNAs or RNAs comprising unusual bases, such as inosine, or modified bases, such as tritylated bases, to name just two examples, are nucleic acid molecules as the term is used herein. It will be appreciated that a great variety of modifications have been made to DNA and RNA that serve many useful purposes known to those of skill in the art. The term “nucleic acid molecule(s)” as it is employed herein embraces such chemically, enzymatically or metabolically modified forms of nucleic acid molecules, as well as the chemical forms of DNA and RNA characteristic of viruses and cells, including, for example, simple and complex cells. “Nucleic acid molecule(s)” also embraces short nucleic acid molecules often referred to as oligonucleotide(s).

An “isolated” nucleic acid molecule is one that is separated from other nucleic acid molecules that are present in the natural source of the nucleic acid. For example, with regard to chromosomal DNA, (e.g. whether chromosomal or episomal) the term “isolated” includes nucleic acid molecules which are separated from flanking DNA sequences with which the DNA is naturally associated. Preferably, an “isolated” nucleic acid molecule is free of sequences which naturally flank the nucleic acid molecule (i.e., sequences located at the 5′ and 3′ ends of the nucleic acid molecule) in the DNA (e.g., chromosomal or episomal) of the organism from which the nucleic acid molecule is derived. As such, isolated DNA is not in its naturally occurring state. For example, in various embodiments, the isolated BLR nucleic acid molecule can contain less than about 5 kb, 4 kb, 3 kb, 2 kb, 1 kb, 0.5 kb, 0.1 kb, or 0.05 kb of nucleotide sequences which naturally flank the nucleic acid molecule in DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. An “isolated” BLR nucleic acid molecule may, however, be linked to other nucleotide sequences that do not normally flank the BLR sequences in genomic DNA (e.g., the BLR nucleotide sequences may be linked to vector sequences). In certain preferred embodiments, an “isolated” nucleic acid molecule, such as a cDNA molecule, also may be free of other cellular material. However, it is not necessary for the BLR nucleic acid molecule to be free of other cellular material to be considered “isolated” (e.g., a BLR DNA molecule separated from other chromosomal DNA and inserted into another bacterial cell would still be considered to be “isolated”).

As used herein, “polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to those of skill in the art. It will be appreciated that the same type of modification may be present in the same or varying degree at several sites in a given polypeptide. Also, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, glycosylation, lipid attachment, sulfation, gamma-carboxylation of glutamic acid residues, hydroxylation and ADP-ribosylation, selenoylation, sulfation, transfer-RNA mediated addition of amino acids to proteins, such as arginylation, and ubiquitination. See, for instance, Proteins—Structure And Molecular Properties, 2^{nd} Ed., T. E. Creighton, W. H. Freeman and Company, New York (1993) and Wold, F., Posttranslational Protein Modifications: Perspectives and Prospects, pgs. 1-12 in Posttranslational Covalent Modification Of Proteins, B. C. Johnson, Ed., Academic Press, New York (1983); Seifter et al., Meth. Enzymol. 182:626-646 (1990) and Rattan et al., Protein Synthesis: Posttranslational Modifications and Aging, Ann. N.Y. Acad. Sci. 663: 48-62 (1992). Polypeptides may be branched or cyclic, with or without branching. Cyclic, branched and branched circular polypeptides may result from post-translational natural processes and may be made by entirely synthetic methods, as well.

As used herein, an “isolated protein” or “isolated polypeptide” refers to a protein or polypeptide that is substantially free of other proteins, polypeptides, cellular material and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the BLR protein is derived, or substantially free from chemical precursors or other chemicals when chemically synthesized. The language “substantially free of cellular material” includes preparations of BLR protein in which the protein is separated from cellular components of the cells from which it is isolated or recombinantly produced. In one embodiment, the language “substantially free of cellular material” includes preparations of BLR protein having less than about 30% (by dry weight) of non-BLR protein (also referred to herein as a “contaminating protein”), more preferably less than about 20% of non-BLR protein, still more preferably less than about 10% of non-BLR protein, and most preferably less than about 5% non-BLR protein. When the BLR protein or biologically active portion thereof is recombinantly produced, it is also preferably substantially free of culture medium, i.e., culture medium represents less than about 20%, more preferably less than about 10%, and most preferably less than about 5% of the volume of the protein preparation.

The language “substantially free of chemical precursors or other chemicals” includes preparations of BLR protein in which the protein is separated from chemical precursors or other chemicals that are involved in the synthesis of the protein. In one embodiment, the language “substantially free of chemical precursors or other chemicals” includes preparations of BLR protein having less than about 30% (by dry weight) of chemical precursors or non-BLR chemicals, more preferably less than about 20% chemical precursors or non-BLR chemicals, still more preferably less than about 10% chemical precursors or non-BLR chemicals, and most preferably less than about 5% chemical precursors or non-BLR chemicals.

Preferred BLR nucleic acid molecules and polypeptides are “naturally occurring.” As used herein, a “naturally-occurring” molecule refers to a BLR molecule having a nucleotide sequence that occurs in nature (e.g., encodes a natural BLR polypeptide). In addition naturally or non-naturally occurring variants of these polypeptides and nucleic acid molecules which retain the same functional activity, e.g., the ability to modulate adaptation to stress and/or virulence in a microbe are included. Such variants can be made, e.g., by mutation using techniques that are known in the art. Alternatively, variants can be chemically synthesized.

As used herein the term “variant(s)” includes nucleic acid molecules or polypeptides that differ in sequence from a reference nucleic acid molecule or polypeptide, but retain essential properties. Changes in the nucleotide sequence of the variant may or may not alter the amino acid sequence of a polypeptide encoded by the reference nucleic acid molecule. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, and/or deletions in any combination. A variant of a nucleic acid molecule or polypeptide may be naturally occurring, such as an allelic variant, or it may be a variant that is not known to occur naturally. Non-naturally occurring variants of nucleic acid molecules and polypeptides may be made by mutagenesis techniques, by direct synthesis, and by other recombinant methods known to skilled artisans.

Preferred BLR nucleic acid molecules and BLR polypeptides are “naturally occurring.” As used herein, a “naturally-occurring” molecule refers to a BLR polypeptide encoded by a nucleotide sequence that occurs in nature (e.g., encodes a natural BLR polypeptide). Such molecules can be obtained from other microbes, e.g., based on their sequence similarity to the BLR molecules described herein.

In addition, naturally or non-naturally occurring variants of these polypeptides and nucleic acid molecules which retain the same functional activity, e.g., the ability to modulate drug resistance in a cell are also within the scope of the invention. Such variants can be made, e.g., by mutation using techniques which are known in the art. Alternatively, variants can be chemically synthesized.

For example, it will be understood that the BLR molecules described herein, also encompass equivalents thereof. For instance, mutant forms of BLR polypeptides which are functionally equivalent to the polypeptide shown in SEQ ID NO:2, (e.g., have the ability to regulate drug resistance) can be made using techniques which are well known in the art. Mutations can include, e.g., at least one of a discrete point mutation which can give rise to a substitution, or by at least one deletion or insertion. For example, random mutagenesis can be used. Mutations can be made by random mutagenesis or using cassette mutagenesis. For the former, the entire coding region of a molecule is mutagenized by one of several methods (e.g., chemical, PCR, doped oligonucleotide synthesis) and that collection of randomly mutated molecules is subjected to selection or screening procedures. In the latter, discrete regions of a protein, corresponding either to defined structural or functional determinants (e.g., the extracellular, transmembrane, or cytoplasmic domain) are subjected to saturating or semi-random mutagenesis and these mutagenized cassettes are re-introduced into the context of the otherwise wild type allele. In one embodiment, PCR mutagenesis can be used. For example, Megaprimer PCR can be used (O. H. Landt, Gene 96:125-128).

As used herein, “heterologous DNA” or “heterologous nucleic acid” includes DNA that does not occur naturally in the cell (e.g., as part of the genome) in which it is present or which is found in a location or locations in the genome that differs from that in which it occurs in nature or which is operatively linked to DNA to which it is not normally linked in nature (i.e., a gene that has been operatively linked to a heterologous promoter). Heterologous DNA is 1) not naturally occurring in a particular position (e.g., at a particular position in the genome) or 2) is not endogenous to the cell into which it is introduced, but has been obtained from another cell. Heterologous DNA can be from the same species or from a different species. Any DNA that one of skill in the art would recognize or consider as heterologous or foreign to the cell in which is expressed is herein encompassed by the term heterologous DNA.

The terms “heterologous protein”, “recombinant protein”, and “exogenous protein” are used interchangeably throughout the specification and refer to a polypeptide which is produced by recombinant DNA techniques, wherein generally, DNA encoding the polypeptide is inserted into a suitable expression vector which is in turn used to transform a host cell to produce the heterologous protein. That is, the polypeptide is expressed from a heterologous nucleic acid molecule.

The term “interact” includes close contact between molecules that results in a measurable effect, e.g., on the conformation and/or activity, of at least one of the molecules involved in the interaction. For example, a first molecule can be said to interact with a second when it inhibits the binding of the second molecule to a target (e.g., a DNA or protein target) to which that second molecule normally binds, or when it alters the activity of the second molecule, e.g., by steric interaction with a domain of the second molecule that mediates its activity. For example, compounds can interact with a BLR nucleic acid molecule and inhibit its transcription or with a BLR polypeptide and alter the activity of the polypeptide.

As used herein, the term “BLR binding polypeptide” includes polypeptides that normally interact with BLR nucleic acid molecules or BLR polypeptides under physiological conditions in a cell, e.g., and altering transcription of a BLR nucleic acid molecule or activity of a BLR polypeptide.

As used herein, the term “drug” includes compounds which reduce the growth, viability, and/or or virulence of a microbe. As used herein, the term “virulence” includes the degree of pathogenicity of an organism. The term virulence encompasses two features of an organism: its infectivity (the ability to colonize a host) and the severity of the disease produced. As used herein, the term “viability” includes the capacity for cell growth. Viable cells may not actively be multiplying, e.g., may be in a quiescent state, but retain the ability to grow when conditions for growth are more favorable. As used herein, the term “growth” includes the ability to multiply, i.e., by cell division or proliferation.

Such drugs include antibiotic agents and non-antibiotic agents. The term “drug” includes antiinfective compounds which are static or cidal for microbes, e.g., an antimicrobial compound which inhibits the growth and/or viability of a microbe. Preferred antiinfective compounds increase the susceptibility of microbes to antibiotics or decrease the infectivity or virulence of a microbe. The term “drug” includes the antimicrobial agents such as disinfectants, antiseptics, and surface delivered compounds. For example, antibiotics or other types of antibacterial compounds, including agents which induce oxidative stress, and organic solvents are included in this term. The term “drug” also includes biocides. The term “biocides” is art recognized and includes an agent that is thought to kill a cell “non-specifically,” or a broad spectrum agent whose mechanism of action is unknown as well as drugs that are known to be target-specific. Examples of biocides include paraben, chlorbutanol, phenol, alkylating agents such as ethylene oxide and formaldehyde, halides, mercurials and other heavy metals, detergents, acids, alkalis, and chlorhexidine. Other biocides agents include: pine oil, quaternary amine compounds such as alkyl dimethyl benzyl ammonium chloride, chloroxylol, chlorhexidine, cyclohexidine, triclocarbon, and disinfectants. The term “bactericidal” refers to an agent that can kill a bacterium; “bacteriostatic” refers to an agent that inhibits the growth of a bacterium.

The term “antibiotic” is art recognized and includes antimicrobial agents synthesized by an organism in nature and isolated from this natural source, and chemically synthesized drugs. The term includes but is not limited to: polyether ionophores such as monensin and nigericin; macrolide antibiotics such as erythromycin and tylosin; aminoglycoside antibiotics such as streptomycin and kanamycin; β-lactam antibiotics (having a β lactam ring) such as penicillin and cephalosporin; and polypeptide antibiotics such as subtilisin and neosporin. Semi-synthetic derivatives of antibiotics, and antibiotics produced by chemical methods are also encompassed by this term. Chemically-derived antimicrobial agents such as isoniazid, trimethoprim, quinolones, fluoroquinolones and sulfa drugs are considered antibacterial drugs, and the term antibiotic includes these. It is within the scope of the screens of the present invention to include compounds derived from natural products and compounds that are chemically synthesized. The term “antibiotic” as used herein includes those antimicrobial agents approved for human use.

The term “antibiotic that affects peptidoglycan synthesis” as used herein.

The phrase “non-antibiotic agent” includes agents that are not art recognized as being antibiotics. Exemplary non-antibiotic agents include, e.g., biocides, disinfectants or antiinfectives. Non antibiotic agents also include compounds incorporated into consumer goods, e.g., for topical use on a subject or as cleaning products. In contrast to the term “biocide,” an antibiotic or an “anti-microbial drug approved for human use” is considered to have a specific molecular target in a microbial cell. Preferably a microbial target of a therapeutic agent is sufficiently different from its physiological counterpart in a subject in need of treatment that the antibiotic or drug has minimal adverse effects on the subject.

As used herein the term “reporter gene” includes any gene that encodes an easily detectable product that is operably linked to a promoter and/or other regulatory region. By operably linked it is meant that under appropriate conditions an RNA polymerase may bind to the promoter of the regulatory region and proceed to transcribe the nucleotide sequence of the reporter gene. In certain embodiments, however, it may be desirable to include other sequences, e.g., transcriptional regulatory sequences, in the reporter gene construct. For example, modulation of the activity of the promoter may be affected by altering the RNA polymerase binding to the promoter region, or, alternatively, by interfering with initiation of transcription or elongation of the mRNA. Thus, sequences which are herein collectively referred to as transcriptional regulatory elements or sequences may also be included in the reporter gene construct. In addition, the construct may include sequences of nucleotides that alter translation of the resulting mRNA, thereby altering the amount of reporter gene product.

As used herein the term “test compound” includes agents which are tested using the assays of the invention to determine whether they modulate the activity of a BLR polypeptide. More than one compound, e.g., a plurality of compounds, can be tested at the same time for their ability to modulate the activity of a BLR polypeptide sequence in a screening assay.

Test compounds that can be assayed in the subject assays include antibiotic and non-antibiotic compounds. In one embodiment, test compounds include candidate detergent or disinfectant compounds. Exemplary compounds which can be screened for activity include, but are not limited to, peptides, non-peptidic compounds, nucleic acid molecules, carbohydrates, small organic molecules (e.g., polyketides), and natural product extract libraries. The term “non-peptidic compound” is intended to encompass compounds that are comprised, at least in part, of molecular structures different from naturally-occurring L-amino acid residues linked by natural peptide bonds. However, “non-peptidic compounds” are intended to include compounds composed, in whole or in part, of peptidomimetic structures, such as D-amino acids, non-naturally-occurring L-amino acids, modified peptide backbones and the like, as well as compounds that are composed, in whole or in part, of molecular structures unrelated to naturally-occurring L-amino acid residues linked by natural peptide bonds. “Non-peptidic compounds” also are intended to include natural products.

As used herein, the term “antibody” is intended to include immunoglobulin molecules and immunologically active portions of immunoglobulin molecules, i.e., molecules that contain an antigen binding site which binds (immunoreacts with) an antigen, such as Fab and F(ab′)₂ fragments, single chain antibodies, intracellular antibodies, scFv, Fd, or other fragments. Preferably, antibodies of the invention bind specifically or substantially specifically to BLR molecules. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody compositions thus typically display a single binding affinity for a particular antigen with which it immunoreacts.

The phrase “specifically” with reference to binding, recognition, or reactivity of antibodies includes antibodies which bind to a naturally occurring BLR molecules, but are substantially unreactive with other unrelated molecules. Preferably, such antibodies bind to a BLR molecule (or its homolog from another species) and bind non-BLR molecules with only background binding. Antibodies specific for BLR molecules from one source may or may not be reactive with BLR molecules from different species. Antibodies specific for naturally occurring BLR molecules may or may not bind to mutant forms of such molecules. Assays to determine affinity and specificity of binding are known in the art, including competitive and non-competitive assays. Assays of interest include ELI SA, RIA, flow cytometry, etc.

The term “microbe” includes microorganisms expressing or made to express a BLR polypeptide. “Microbes” are of some economic importance, e.g., are environmentally important or are important as human pathogens. For example, in one embodiment microbes cause environmental problems, e.g., fouling or spoilage, or perform useful functions such as breakdown of plant matter. In another embodiment, microbes are organisms that live in or on mammals and are medically important. Preferably, microbes are unicellular and include bacteria, fungi, or protozoa. In another embodiment, microbes suitable for use in the invention are multicellular, e.g., parasites or fungi. In preferred embodiments, microbes are pathogenic for humans, animals, or plants. Microbes may be used as intact cells or as sources of materials for assays as described herein.

II. Compositions which Modulate Antibiotic Resistance

A. Nucleic Acid Molecules

In one aspect, the invention provides isolated nucleic acid molecules comprising or consisting essentially of Beta Lactam-358 (BLR) nucleotide sequences. In another aspect, the invention provides nucleic acid molecules consisting of BLR nucleotide sequences. An exemplary nucleotide sequence of a BLR nucleic acid molecule is shown in SEQ ID NO:1.

BLR nucleotide sequences have structural similarity (e.g., to the sequence shown in SEQ ID NO:1) and, preferably, encode BLR polypeptides having a BLR polypeptide activity. For example, in one embodiment, a BLR polypeptide is capable of modulating resistance to a wide variety of drugs. In particularly preferred embodiments, BLR nucleic acid molecules modulate resistance to antibiotics, preferably antibiotics that inhibit peptidoglycan synthesis.

In one embodiment, a BLR nucleotide sequence is isolated from an intergenic region of a microbial chromosome. Preferably a BLR nucleotide sequence is at least about 500, 450, 400, or 350 bp in length and encodes a polypeptide of less than about 150, 100, 75, or 50 amino acids in length. In one embodiment, portions of a BLR nucleotide sequence which encode a polypeptide having a BLR polypeptide activity are also provided for.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid molecule and the amino acid sequence encoded by that nucleic acid molecule, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp,D) | GAC, GAT |
| Cysteine (Cys, C) | TGC, TGT |
| GLutamic acid (Glu,E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe,F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA molecule coding for a BLR polypeptide of the invention (or a portion thereof) can be used to derive the BLR amino acid sequence, using the genetic code to translate the DNA or RNA molecule into an amino acid sequence. Likewise, for any BLR-amino acid sequence, corresponding nucleotide sequences that can encode a BLR protein can be deduced from the genetic code (which, because of its redundancy, will produce multiple nucleic acid sequences for any given amino acid sequence). Thus, description and/or disclosure herein of a BLR related nucleotide sequence should be considered to also include description and/or disclosure of the amino acid sequence encoded by the nucleotide sequence. Similarly, description and/or disclosure of a BLR amino acid sequence herein should be considered to also include description and/or disclosure of all possible nucleotide sequences that can encode the amino acid sequence.

One aspect of the invention pertains to isolated nucleic acid molecules that encode BLR proteins or biologically active portions thereof, as well as nucleic acid fragments sufficient for use as hybridization probes to identify BLR-encoding nucleic acids (e.g., BLR mRNA) and fragments for use as PCR primers for the amplification or mutation of BLR nucleic acid molecules. It will be understood that in discussing the uses of BLR nucleic acid molecules, e.g., as shown in SEQ. ID NO:1, that fragments of such nucleic acid molecules as well as full length BLR nucleic acid molecules can be used.

A nucleic acid molecule of the present invention, e.g., a nucleic acid molecule having the nucleotide sequence of SEQ ID NO:1, or a portion thereof, can be isolated using standard molecular biology techniques and the sequence information provided herein. For example, using all or portion of the nucleic acid sequence of SEQ ID NO:1 as a hybridization probe, BLR nucleic acid molecules can be isolated using standard hybridization and cloning techniques (e.g., as described in Sambrook, J., Fritsh, E. F., and Maniatis, T. Molecular Cloning: A Laboratory Manual. 2nd, ed, Cold Spring Harbor Laboratory, Cold Spring Harbor Laboratory Press, Cold Spring Harbor, N.Y., 1989). Moreover, a nucleic acid molecule encompassing all or a portion of SEQ ID NO:1 can be isolated by the polymerase chain reaction (PCR) using synthetic oligonucleotide primers designed based upon the sequence of SEQ ID NO:1 respectively.

A nucleic acid molecule of the invention can be amplified using cDNA, mRNA or alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, oligonucleotides corresponding to BLR nucleotide sequences can be prepared by standard synthetic techniques, e.g., using an automated DNA synthesizer.

In another preferred embodiment, an isolated nucleic acid molecule of the invention comprises a nucleic acid molecule which is a complement of the nucleotide sequence shown in SEQ ID NO:1 or a portion of the nucleotide sequence. A nucleic acid molecule having a nucleotide sequence which is complementary to the nucleotide sequence shown in SEQ ID NO:1 is one which is sufficiently complementary to the nucleotide sequence shown in SEQ ID NO:1 such that it can hybridize to the nucleotide sequence shown in SEQ ID NO:1, thereby forming a stable duplex.

In addition to the nucleic acid molecule shown in SEQ ID NO: 1, other BLR nucleotide sequences of the invention are “structurally related” (i.e., share sequence identity with) the BLR nucleotide sequence shown in SEQ ID NO:1. Such sequence similarity can be shown, e.g., by optimally aligning the BLR nucleotide sequence with a putative BLR nucleotide sequence using an alignment program for purposes of comparison and comparing corresponding positions. In a preferred embodiment, an isolated nucleic acid molecule of the invention comprises the nucleotide sequence shown in SEQ ID NO:1.

In still another preferred embodiment, an isolated nucleic acid molecule of the present invention comprises a nucleotide sequence which is at least about 30%, 40%, 50%, 55%, 60%, 65%, 70%, 75%, 80%, 85%, 90%, 95%, 98% or more homologous to a nucleotide sequence (e.g., to the entire length of a nucleotide sequence) of a BLR shown in SEQ ID NO:1 or a portion thereof.

In other embodiments, a nucleic acid molecule of the invention has at least 50%, 60%, 70% identity, more preferably 80% identity, and even more preferably 90% identity with a nucleic acid molecule comprising: at least about 100, 150, 200, 250, 300, or at about 350 contiguous nucleotides of SEQ ID NO: 1.

Sequence similarity can be shown, e.g., by optimally aligning BLR nucleotide or amino acid sequences for purposes of comparison using an alignment program and comparing corresponding positions of the sequences. To determine the degree of similarity between sequences, they can be aligned for optimal comparison purposes (e.g., gaps may be introduced in the sequence of one polypeptide or nucleic acid molecule for optimal alignment with the other polypeptide or nucleic acid molecule with which they are to be compared). The amino acid residues or bases at a given position are then compared with the corresponding amino acid residue or base in the sequence with which they are being compared. When a position in one sequence is occupied by the same amino acid residue or by the same base as the corresponding position in the other sequence, then the sequences are identical at that position. If amino acid residues are not identical, they may be similar. As used herein, an amino acid residue is “similar” to another amino acid residue if the two amino acid residues are members of the same family of residues having similar side chains. Families of amino acid residues having similar side chains have been defined in the art (see, for example, Altschul et al. 1990. J. Mol. Biol. 215:403) including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan.) The degree (percentage) of similarity between sequences, therefore, is a function of the number of identical or similar positions shared by two sequences (i. e., % homology=# of identical or similar positions/total # of positions×100). Alignment strategies are well known in the art; see, for example, Altschul et al. supra for optimal sequence alignment.

Preferably, BLR polypeptides share some amino acid sequence similarity with a polypeptide of SEQ ID NO:2, encoded by a BLR gene set forth in SEQ ID NO:1. The nucleic acid and/or amino acid sequences of an BLR gene or polypeptide (e.g., as provided above) can be used as “query sequence” to perform a search against databases (e.g., either public or private such as http://www.tigr.org) to, for example, identify other BLR genes (or polypeptides) having related sequences. For example, such searches can be performed, e.g., using the NBLAST and XBLAST programs (version 2.0) of Altschul, et al. (1990) J. Mol. Biol. 215:403-10. BLAST nucleotide searches can be performed with the NBLAST program, score 100, wordlength=12 to obtain nucleotide sequences homologous to the above BLR nucleic acid molecules. BLAST polypeptide searches can be performed with the XBLAST program, score=50, wordlength 3 to obtain amino acid sequences homologous to BLR polypeptide molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

However, it will be understood that the level of sequence identity among microbial genes, even though members of the same family, is not necessarily high. This is particularly true in the case of divergent genomes where the level of sequence identity may be low, e.g., less than 20% (e.g., B. burgdorferi as compared e.g., to B. subtilis). Accordingly, structural similarity among BLR-molecules can also be determined based on “three-dimensional correspondence” of amino acid residues. As used herein, the language “three-dimensional correspondence” is meant to include residues which spatially correspond, e.g., are in the same functional position of a BLR polypeptide member as determined, e.g., by x-ray crystallography, but which may not correspond when aligned using a linear alignment program. The language “three-dimensional correspondence” also includes residues which perform the same function, e.g., bind to DNA or bind the same cofactor, as determined, e.g., by mutational analysis.

Nucleic acid molecules that differ from SEQ ID NO: 1 due to degeneracy of the genetic code, and thus encode the same BLR protein as that encoded by SEQ ID NO: 1 are encompassed by the invention. Accordingly, in another embodiment, an isolated nucleic acid molecule of the invention has a nucleotide sequence encoding a protein having an amino acid sequence shown in SEQ ID NO: 2.

In addition to the BLR nucleotide sequence shown in SEQ ID NO:1, it will be appreciated by those skilled in the art that DNA sequence polymorphisms that lead to changes in the amino acid sequences of a given BLR polypeptide may exist within a population of organisms. Such nucleotide variations and resulting amino acid polymorphisms in BLR genes that are the result of natural allelic variation and that do not alter the functional activity of a BLR polypeptide are intended to be within the scope of the invention.

Moreover, nucleic acid molecules encoding other BLR polypeptides and, thus, which have a nucleotide sequence which differs from the BLR sequence of SEQ ID NO:1 are intended to be within the scope of the invention. Moreover, nucleic acid molecules encoding BLR proteins from different species, and thus which have a nucleotide sequence which differs from the BLR sequence of SEQ ID NO:1 are intended to be within the scope of the invention.

BLR nucleic acid molecules can also be identified as being structurally similar to the exemplary BLR gene set forth herein based on their ability to hybridize to the nucleic acid molecule set forth in SEQ ID NO:1 under stringent conditions. For example, a BLR DNA can be isolated from a DNA library using all or portion of SEQ ID NO:1 as a hybridization probe and standard hybridization techniques (e.g., as described in Sambrook, J., et al. Molecular Cloning: A Laboratory Manual. 2nd, ed., Cold Spring Harbor Laboratory, Cold Spring Harbor, N.Y., 1989).

As used herein, the term “hybridizes under stringent conditions” is intended to describe conditions for hybridization and washing under which nucleotide sequences at least 30%, 40%, 50%, or 60% homologous to each other typically remain hybridized to each other. Preferably, the conditions are such that sequences at least about 70%, more preferably at least about 80%, even more preferably at least about 85% or 90% homologous to each other typically remain hybridized to each other. Preferably, an isolated nucleic acid molecule of the invention that hybridizes under stringent conditions to the sequence of SEQ ID NO:1 or its complement corresponds to a naturally-occurring nucleic acid molecule. Such stringent conditions are known to those skilled in the art and can be found e.g., in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50-65° C. Conditions for hybridizations are largely dependent on the melting temperature Tm that is observed for half of the molecules of a substantially pure population of a double-stranded nucleic acid. Tm is the temperature in ° C. at which half the molecules of a given sequence are melted or single-stranded. For nucleic acids of sequence 11 to 23 bases, the Tm can be estimated in degrees C as 2 (number of A+T residues)+4(number of C+G residues). Hybridization or annealing of nucleic acid molecules should be conducted at a temperature lower than the Tm, e.g., 15° C., 20° C., 25° C. or 30° C. lower than the Tm. The effect of salt concentration (in M of NaCl) can also be calculated, see for example, Brown, A., “Hybridization” pp. 503-506, in The Encyclopedia of Molec. Biol., J. Kendrew, Ed., Blackwell, Oxford (1994).

Moreover, the nucleic acid molecules of the invention can comprise only a portion of the nucleic acid sequence of SEQ ID NO:1 for example a fragment which can be used as a probe or primer or a fragment encoding a biologically active portion of a BLR protein. The nucleotide sequence determined from the cloning of BLR genes allows for the generation of probes and primers designed for use in identifying and/or cloning other BLR polypeptides, as well as BLR homologues from other species. The probe/primer typically comprises a substantially purified oligonucleotide. In one embodiment, the oligonucleotide comprises a region of nucleotide sequence that hybridizes under stringent conditions to at least about 12 or 15, preferably about 20 or 25, more preferably about 30, 35, 40, 45, 50, 55, 60, 65, 75, or 100 consecutive nucleotides of a sense sequence of SEQ ID NO:1 or of a naturally occurring allelic variant or mutant of SEQ ID NO:1. In another embodiment, a nucleic acid molecule of the present invention comprises a nucleotide sequence which is at least about 200, 250, 300, or 350 nucleotides in length and hybridizes under stringent hybridization conditions to a nucleic acid molecule of SEQ ID NO:1 or the complement thereof.

Moreover, a nucleic acid molecule encompassing all or a portion of a BLR gene can be isolated by the polymerase chain reaction using oligonucleotide primers designed based upon the sequence of SEQ ID NO: 1. For example, RNA can be isolated from cells (e.g., by the guanidinium-thiocyanate extraction procedure of Chirgwin et al. (1979) Biochemistry 18: 5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for PCR amplification can be designed based upon the nucleotide sequence shown in SEQ ID NO: 1. A nucleic acid molecule of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a BLR nucleotide sequence can be prepared by standard synthetic techniques, e.g., using an automated DNA synthesizer.

In addition to naturally-occurring allelic variants of BLR sequences that may exist in the population, the skilled artisan will further appreciate that minor changes may be introduced by mutation into nucleotide sequences, e.g., of SEQ ID NO: 1, thereby leading to changes in the amino acid sequence of the encoded polypeptide, without altering the functional activity of a BLR polypeptide. For example, nucleotide substitutions leading to amino acid substitutions at “non-essential” amino acid residues may be made in the sequence of SEQ ID NO: 1. A “non-essential” amino acid residue is a residue that can be altered from the wild-type sequence of a BLR nucleic acid molecule (e.g., the sequence of SEQ ID NO: 1) without altering the functional activity of a BLR molecule. Exemplary residues which are non-essential and, therefore, amenable to substitution, can be identified by one of ordinary skill in the art, e.g., by performing an amino acid alignment of BLR-molecules and determining residues that are not conserved or by alanine scanning mutagenesis. Such residues, because they have not been conserved, are more likely amenable to substitution.

Accordingly, another aspect of the invention pertains to nucleic acid molecules encoding BLR proteins that contain changes in amino acid residues that are not essential for a BLR activity. Such BLR proteins differ in amino acid sequence from SEQ ID NO: 2 yet retain an inherent BLR activity. An isolated nucleic acid molecule encoding a non-natural variant of a BLR polypeptide can be created by introducing one or more nucleotide substitutions, additions or deletions into the nucleotide sequence of SEQ ID NO: 1 such that one or more amino acid substitutions, additions or deletions are introduced into the encoded polypeptide. Mutations can be introduced into SEQ ID NO: 1 by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in a BLR polypeptide is preferably replaced with another amino acid residue from the same side chain family.

Alternatively, in another embodiment, mutations can be introduced randomly along all or part of a BLR coding sequence, such as by saturation mutagenesis, and the resultant mutants can be screened for activity, to identify mutants that retain functional activity. Following mutagenesis, the encoded a BLR mutant polypeptide can be expressed recombinantly in a host cell and the functional activity of the mutant polypeptide can be determined using assays available in the art for assessing a BLR activity.

Yet another aspect of the invention pertains to isolated nucleic acid molecules encoding a BLR fusion polypeptide. Such nucleic acid molecules, comprising at least a first nucleotide sequence encoding a full-length BLR protein, polypeptide or peptide having a BLR activity operatively linked to a second nucleotide sequence encoding a non-BLR protein, polypeptide or peptide, can be prepared by standard recombinant DNA techniques.

In addition to the nucleic acid molecules encoding BLR proteins described above, another aspect of the invention pertains to isolated nucleic acid molecules which are antisense thereto. An “antisense” nucleic acid molecule comprises a nucleotide sequence which is complementary to a “sense” nucleic acid molecule encoding a polypeptide, e.g., complementary to the coding strand of a double-stranded cDNA molecule or complementary to an mRNA sequence. Accordingly, an antisense nucleic acid molecule can hydrogen bond to a sense nucleic acid molecule. The antisense nucleic acid molecule can be complementary to an entire BLR coding strand, or only to a portion thereof. In one embodiment, an antisense nucleic acid molecule is antisense to a “coding region” of the coding strand of a nucleotide sequence encoding BLR. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding BLR. The term “noncoding region” refers to 5′ and 3′ sequences which flank the coding region that are not translated into amino acids.

With the coding strand sequences encoding BLR molecule disclosed herein, antisense nucleic acids of the invention can be designed according to the rules of Watson and Crick base pairing. The antisense nucleic acid molecule can be complementary to the entire coding region of BLR mRNA, but more preferably is an oligonucleotide which is antisense to only a portion of the coding or noncoding region of BLR mRNA. For example, the antisense oligonucleotide can be complementary to the region surrounding the translation start site of BLR mRNA. An antisense oligonucleotide can be, for example, about 5, 10, 15, 20, 25, 30, 35, 40, 45 or 50 nucleotides in length. An antisense nucleic acid molecule of the invention can be constructed using chemical synthesis and enzymatic ligation reactions using procedures known in the art. For example, an antisense nucleic acid molecule (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid molecule can be produced biologically using an expression vector into which a nucleic acid molecule has been subcloned in an antisense orientation (i.e., RNA transcribed from the inserted nucleic acid molecule will be of an antisense orientation to a target nucleic acid molecule of interest).

The antisense nucleic acid molecules of the invention are typically administered to a subject or generated in situ such that they hybridize with or bind to cellular nucleic acid molecules to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention include direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215 :327-330).

In still another embodiment, an antisense nucleic acid molecule of the invention is a ribozyme. Ribozymes are catalytic RNA molecules with ribonuclease activity which are capable of cleaving a single-stranded nucleic acid molecule, such as an mRNA, to which they have a complementary region. Thus, ribozymes (e.g., hammerhead ribozymes (described in Haselhoff and Gerlach (1988) Nature 334:585-591)) can be used to catalytically cleave BLR mRNA transcripts to thereby inhibit translation of BLR mRNA. A ribozyme having specificity for a BLR-encoding nucleic acid molecule can be designed based upon the nucleotide sequence of SEQ ID NO:1. For example, a derivative of a Tetrahymena L-19 IVS RNA can be constructed in which the nucleotide sequence of the active site is complementary to the nucleotide sequence to be cleaved in a BLR-encoding mRNA. See, e.g., Cech et al. U.S. Pat. No. 4,987,071; and Cech et al. U.S. Pat. No. 5,116,742. Alternatively, BLR mRNA can be used to select a catalytic RNA having a specific ribonuclease activity from a pool of RNA molecules. See, e.g., Bartel, D. and Szostak, J. W. (1993) Science 261:1411-1418.

Alternatively, gene expression can be inhibited by targeting nucleotide sequences complementary to the regulatory region of BLR (e.g., the BLR promoter and/or enhancers) to form triple helical structures that prevent transcription of the BLR gene in target cells. See generally, Helene, C. (1991) Anticancer Drug Des. 6(6):569-84; Helene, C. et al. (1992) Ann. N.Y. Acad. Sci. 660:27-36; and Maher, L. J. (1992) Bioassays 14(12):807-15.

In yet another embodiment, the BLR nucleic acid molecules of the present invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

PNAs of BLR nucleic acid molecules can be used in therapeutic and diagnostic applications. For example, PNAs can be used as antisense or antigene agents for sequence-specific modulation of gene expression by, for example, inducing transcription or translation arrest or inhibiting replication. PNAs of BLR nucleic acid molecules can also be used in the analysis of single base pair mutations in a gene, (e.g., by PNA-directed PCR clamping); as ‘artificial restriction enzymes’ when used in combination with other enzymes, (e.g., S1 nucleases (Hyrup B. (1996) supra)); or as probes or primers for DNA sequencing or hybridization (Hyrup B. et al. (1996) supra; Perry-O'Keefe supra).

In another embodiment, PNAs of BLR molecules can be modified, (e.g., to enhance their stability or cellular uptake), by attaching lipophilic or other helper groups to PNA, by the formation of PNA-DNA chimeras, or by the use of liposomes or other techniques of drug delivery known in the art. For example, PNA-DNA chimeras of BLR nucleic acid molecules can be generated which may combine the advantageous properties of PNA and DNA. Such chimeras allow DNA recognition enzymes, (e.g., RNAse H and DNA polymerases), to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup B. (1996) supra). The synthesis of PNA-DNA chimeras can be performed as described in Hyrup B. (1996) supra and Finn P. J. et al. (1996) Nucleic Acids Res. 24 (17): 3357-63. For example, a DNA chain can be synthesized on a solid support using standard phosphoramidite coupling chemistry and modified nucleoside analogs, e.g., 5′-(4-methoxytrityl)amino-5′-deoxy-thymidine phosphoramidite, can be used as a bridge between the PNA and the 5′ end of DNA (Mag, M. et al. (1989) Nucleic Acid Res. 17: 5973-88). PNA monomers are then coupled in a stepwise manner to produce a chimeric molecule with a 5′ PNA segment and a 3′ DNA segment (Finn P. J. et al. (1996) supra). Alternatively, chimeric molecules can be synthesized with a 5′ DNA segment and a 3′ PNA segment (Peterser, K. H. et al. (1975) Bioorganic Med. Chem. Lett. 5: 1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. US. 86:6553-6556; Lemaitre et al. (1987) Proc. Natl. Acad. Sci. USA 84:648-652; PCT Publication No. WO88/098 10) or the blood-brain barrier (see, e.g., PCT Publication No. WO89/10134). In addition, oligonucleotides can be modified with hybridization-triggered cleavage agents (See, e.g., Krol et al. (1988) Bio-Techniques 6:958-976) or intercalating agents. (See, e.g., Zon (1988) Pharm. Res. 5:539-549). To this end, the oligonucleotide may be conjugated to another molecule, (e.g., a peptide, hybridization triggered cross-linking agent, transport agent, or hybridization-triggered cleavage agent).

B. BLR Polypeptides, Fragments thereof, and Anti-BLR Antibodies

One aspect of the invention pertains to isolated BLR polypeptides, and biologically active portions thereof, as well as polypeptide fragments suitable for use as immunogens to raise anti-BLR antibodies.

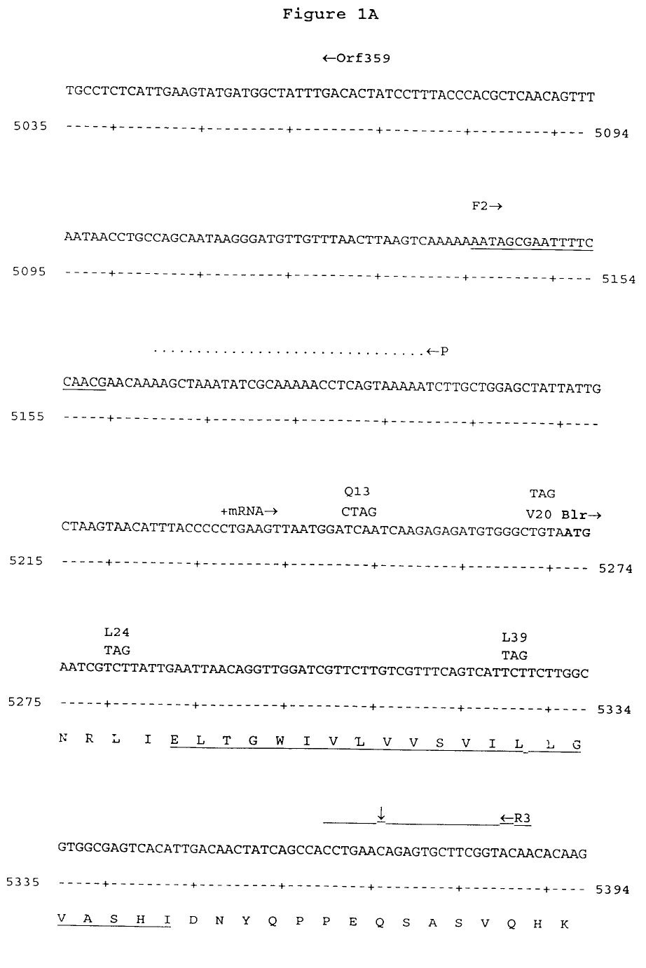

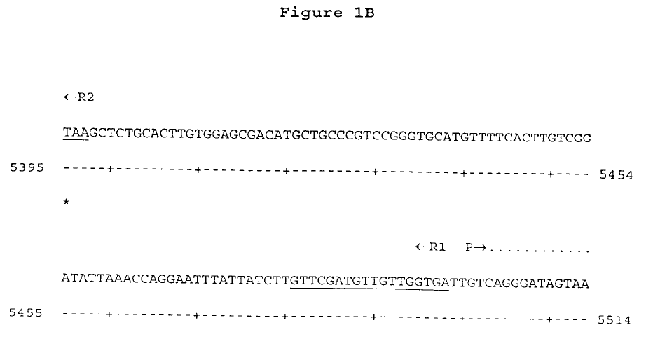

Full length BLR polypeptides comprise several domains. There is a putative transmembrane domain in a BLR polypeptide (e.g., as shown upstream from the point of fusion in FIG. 1). The carboxy terminus of BLR polypeptides is predicted to be intracellular. Accordingly, in one embodiment, a polypeptide comprising an active amino and/or carboxy terminus of BLR can be used in a screening assay. For example, compounds can be tested for their ability to modulate, (e.g., upregulate or downregulate) a BLR-gene, translation of a BLR polypeptide, and/or the ability of a BLR polypeptide to modulate drug resistance.

In one embodiment, native BLR polypeptides can be isolated from cells or tissue sources by an appropriate purification scheme using standard polypeptide purification techniques. In another embodiment, BLR polypeptides are produced by recombinant DNA techniques. Alternative to recombinant expression, a BLR polypeptide or polypeptide can be synthesized chemically using standard peptide synthesis techniques. It will be understood that in discussing the uses of BLR polypeptides, e.g., as shown in SEQ. ID NO:2, that fragments of such polypeptides that are not full length BLR polypeptides as well as full length BLR polypeptides can be used.

Preferably, the BLR polypeptides comprise the amino acid sequence encoded by SEQ ID NO:1 or a portion thereof. In another preferred embodiment, the polypeptide comprises the amino acid sequence of SEQ ID NO:2 or a portion thereof.

Preferred BLR polypeptides are naturally occurring. In other embodiments, the polypeptide has at least 30%, 40%, 50%, at least 60% amino acid identity, more preferably 70% amino acid identity, more preferably 80%, and even more preferably, 90% or 95% amino acid identity with the amino acid sequence shown in SEQ ID NO: 2 or a portion thereof. Preferred portions of BLR polypeptide molecules are biologically active, i.e., encode a portion of the BLR polypeptide having the ability to modulate drug resistance in a cell.

In addition, naturally or non-naturally occurring variants of these polypeptides and nucleic acid molecules which retain the same functional activity, e.g., the ability to modulate drug resistance in a cell are also within the scope of the invention. Such variants can be made, e.g., by mutation using techniques which are known in the art. Alternatively, variants can be chemically synthesized.

For example, it will be understood that the BLR polypeptides described herein also encompass equivalents thereof. For instance, mutant forms of BLR polypeptides which are functionally equivalent, (e.g., have the ability to regulate drug resistance) can be made using techniques which are well known in the art. Mutations can include, e.g., at least one of a discrete point mutation which can give rise to a substitution, or by at least one deletion or insertion. For example, random mutagenesis can be used. Mutations can be made by random mutagenesis or using cassette mutagenesis. For the former, the entire coding region of a molecule is mutagenized by one of several methods (chemical, PCR, doped oligonucleotide synthesis) and that collection of randomly mutated molecules is subjected to selection or screening procedures. In the latter, discrete regions of a polypeptide, corresponding either to defined structural or functional determinants (e.g., the extracellular, transmembrane, or cytoplasmic domain) are subjected to saturating or semi-random mutagenesis and these mutagenized cassettes are re-introduced into the context of the otherwise wild type allele. In one embodiment, PCR mutagenesis can be used. For example, Megaprimer PCR can be used (O. H. Landt, Gene 96:125-128).

In addition to full length BLR polypeptides, fragments of BLR polypeptides and their use are also within the scope of the invention. As used herein, a fragment of a BLR polypeptide refers to a portion of a full-length BLR polypeptide which is useful in a screening assay to identify compounds which modulate a biological activity of a BLR polypeptide (e.g., alter the ability of a BLR polypeptide to influence drug resistance in a microbe). Accordingly, isolated BLR polypeptides for use in the instant screening assays can be full length BLR polypeptides or fragments thereof. Thus, an isolated BLR polypeptide can comprise, consist essentially of, or consist of an amino acid sequence derived from the full length amino acid sequence of a BLR polypeptide, provided that it retains a BLR polypeptide activity.

Portions of the above described polypeptides suitable for use in the claimed assays, such as those which retain their function (e.g., the ability to modulate drug resistance, the ability to modulate drug efflux from a cell, or those which are critical for binding to other molecules (such as DNA, proteins, or compounds) can be easily determined by one of ordinary skill in the art, e.g., using standard truncation or mutagenesis techniques and used in the instant assays. Exemplary techniques are described by Gallegos et al. (1996. J. Bacteriol. 178:6427). In addition, biologically active portions of a BLR polypeptide include peptides comprising amino acid sequences sufficiently homologous to or derived from the amino acid sequence of the BLR polypeptide, which include fewer amino acids than the full length BLR polypeptides, and exhibit at least one activity of a BLR polypeptide are also the subject of the invention.

Other fragments include, for example, truncation polypeptides having a portion of an amino acid sequence shown in SEQ ID NO:2, or of variants thereof, such as a continuous series of residues that includes the amino terminus, or a continuous series of residues that includes the carboxyl terminus. Degradation forms of the polypeptides of the invention in a host cell are also preferred. Further preferred are fragments characterized by structural or functional attributes such as fragments that comprise alpha-helix and alpha-helix forming regions, beta-sheet and beta-sheet-forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100). The percent identity between the two sequences is a function of the number of identical positions shared by the sequences, taking into account the number of gaps, and the length of each gap, which are introduced for optimal alignment of the two sequences. As used herein amino acid or nucleic acid “identity” is equivalent to amino acid or nucleic acid “homology”.

The comparison of sequences and determination of percent identity between two sequences can be accomplished using a mathematical algorithm. A non-limiting example of a mathematical algorithm utilized for comparison of sequences is the algorithm of Karlin and Altschul (1990) Proc. Natl. Acad. Sci. USA 87:2264, modified as in Karlin and Altschul (1993) Proc. Natl. Acad. Sci. USA 90:5873. Such an algorithm is incorporated into the NBLAST and XBLAST programs (version 2.0) of Altschul, et al. (1990) J. Mol. Biol. 215:403. BLAST nucleotide searches can be performed with the NBLAST program score=100, wordlength=12 to obtain nucleotide sequences homologous to the nucleic acid molecules of the invention. BLAST polypeptide searches can be performed with the XBLAST program, score=50, wordlength=3 to obtain amino acid sequences homologous to the polypeptide molecules of the invention. To obtain gapped alignments for comparison purposes, Gapped BLAST can be utilized as described in Altschul et al., (1997) Nucleic Acids Research 25(17):3389. When utilizing BLAST and Gapped BLAST programs, the default parameters of the respective programs (e.g., XBLAST and NBLAST) can be used. See http://www.ncbi.nlm.nih.gov. Another preferred, non-limiting algorithm utilized for the comparison of sequences is the algorithm of Myers and Miller, CABIOS (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

Another non-limiting example of a mathematical algorithm utilized for the alignment of polypeptide sequences is the Lipman-Pearson algorithm (Lipman and Pearson (1985) Science 227:1435). When using the Lipman-Pearson algorithm, a PAM250 weight residue table, a gap length penalty of 12, a gap penalty of 4, and a Kutple of 2 can be used. A preferred, non-limiting example of a mathematical algorithm utilized for the alignment of nucleic acid sequences is the Wilbur-Lipman algorithm (Wilbur and Lipman (1983) Proc. Natl. Acad. Sci. USA 80:726). When using the Wilbur-Lipman algorithm, a window of 20, gap penalty of 3, Ktuple of 3 can be used. Both the Lipman-Pearson algorithm and the Wilbur-Lipman algorithm are incorporated, for example, into the MEGALIGN program (e.g., version 3.1.7) which is part of the DNASTAR sequence analysis software package.

Additional algorithms for sequence analysis are known in the art, and include ADVANCE and ADAM., described in Torelli and Robotti (1994) Comput. Appl. Biosci. 10:3; and FASTA, described in Pearson and Lipman (1988) PNAS 85:2444.

In a preferred embodiment, the percent identity between two amino acid sequences is determined using the GAP program in the GCG software package, using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna. CMP matrix and a gap weight of 40, 50, 60, 70, or 80 and a length weight of 1, 2, 3, 4, 5, or 6.

Protein alignments can also be made using the Geneworks global polypeptide alignment program (e.g., version 2.5.1) with the cost to open gap set at 5, the cost to lengthen gap set at 5, the minimum diagonal length set at 4, the maximum diagonal offset set at 130, the consensus cutoff set at 50% and utilizing the Pam 250 matrix.

The nucleic acid and polypeptide sequences of the present invention can further be used as a “query sequence” to perform a search against public databases to, for example, identify other members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to BLR nucleic acid molecules of the invention. BLAST polypeptide searches can be performed with the XBLAST program, score=50, wordlength=3 to obtain amino acid sequences homologous to BLR polypeptide molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. For example, the nucleotide sequences of the invention can be analyzed using the default Blastn matrix 1-3 with gap penalties set at: existence 11 and extension 1. The amino acid sequences of the invention can be analyzed using the default settings: the Blosum62 matrix with gap penalties set at existence 11 and extension 1. See http://www.ncbi.nlm.nih.gov.

The invention also provides BLR chimeric or fusion polypeptides. As used herein, a BLR “chimeric polypeptide” or “fusion polypeptide” comprises a BLR polypeptide operatively linked to a non-BLR polypeptide. An “BLR polypeptide” refers to a polypeptide having an amino acid sequence corresponding to BLR polypeptide, whereas a “non-BLR polypeptide” refers to a polypeptide having an amino acid sequence corresponding to a polypeptide which is not substantially homologous to the BLR polypeptide, e.g., a polypeptide which is different from the BLR polypeptide and which is derived from the same or a different organism. Within a BLR fusion polypeptide the BLR polypeptide can correspond to all or a portion of a BLR polypeptide. In a preferred embodiment, a BLR fusion polypeptide comprises at least one biologically active portion of a BLR polypeptide. Within the fusion polypeptide, the term “operatively linked” is intended to indicate that the BLR polypeptide and the non-BLR polypeptide are fused in-frame to each other. The non-BLR polypeptide can be fused to the N-terminus or C-terminus of the BLR polypeptide.

For example, in one embodiment, the fusion polypeptide is a GST-BLR member fusion polypeptide in which the BLR member sequences are fused to the C-terminus of the GST sequences. In another embodiment, the fusion polypeptide is a BLR-HA fusion polypeptide in which the BLR member nucleotide sequence is inserted in a vector such as pCEP4-HA vector (Herrscher, R. F. et al. (1995) Genes Dev. 9:3067-3082) such that the BLR member sequences are fused in frame to an influenza hemagglutinin epitope tag. Such fusion polypeptides can facilitate the purification of a recombinant BLR polypeptide.

Fusion polypeptides and peptides produced by recombinant techniques may be secreted and isolated from a mixture of cells and medium containing the polypeptide or peptide. Alternatively, the polypeptide or peptide may be retained cytoplasmically and the cells harvested, lysed and the polypeptide isolated. A cell culture typically includes host cells, media and other byproducts. Suitable media for cell culture are well known in the art. Polypeptides can be isolated from cell culture media, host cells, or both using techniques known in the art for purifying polypeptides and peptides. Techniques for transfecting host cells and purifying polypeptides and peptides are known in the art.

Preferably, a BLR fusion polypeptide of the invention is produced by standard recombinant DNA techniques. For example, DNA fragments coding for the different polypeptide sequences are ligated together in-frame in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide or an HA epitope tag). A BLR encoding nucleic acid molecule can be cloned into such an expression vector such that the fusion moiety is linked in-frame to the BLR polypeptide.

In another embodiment, the fusion polypeptide is a BLR polypeptide containing a heterologous signal sequence at its N-terminus. In certain host cells (e.g., mammalian host cells), expression and/or secretion of BLR can be increased through use of a heterologous signal sequence. The BLR fusion polypeptides of the invention can be incorporated into pharmaceutical compositions and administered to a subject in vivo. Use of BLR fusion polypeptides may be useful therapeutically for the treatment of infection. Moreover, the BLR-fusion polypeptides of the invention can be used as immunogens to produce anti-BLR antibodies in a subject.

Preferably, a BLR chimeric or fusion polypeptide of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A BLR-encoding nucleic acid molecule can be cloned into such an expression vector such that the fusion moiety is linked in-frame to the BLR polypeptide.

The present invention also pertains to variants of the BLR polypeptides which function as either BLR agonists (mimetics) or as BLR antagonists. Variants of the BLR polypeptides can be generated by mutagenesis, e.g., discrete point mutation or truncation of a BLR polypeptide. An agonist of the BLR polypeptides can retain substantially the same, or a subset, of the biological activities of the naturally occurring form of a BLR polypeptide. An antagonist of a BLR polypeptide can inhibit one or more of the activities of the naturally occurring form of the BLR polypeptide by, for example, competitively modulating a cellular activity of a BLR polypeptide. Thus, specific biological effects can be elicited by treatment with a variant of limited function. In one embodiment, treatment of a subject with a variant having a subset of the biological activities of the naturally occurring form of the polypeptide has fewer side effects in a subject relative to treatment with the naturally occurring form of the BLR polypeptide.

In one embodiment, the invention pertains to derivatives of BLR which may be formed by modifying at least one amino acid residue of BLR by oxidation, reduction, or other derivatization processes known in the art.

In one embodiment, variants of a BLR polypeptide which function as either BLR agonists (mimetics) or as BLR antagonists can be identified by screening combinatorial libraries of mutants, e.g., truncation mutants, of a BLR polypeptide for BLR polypeptide agonist or antagonist activity. In one embodiment, a variegated library of BLR variants is generated by combinatorial mutagenesis at the nucleic acid level and is encoded by a variegated gene library. A variegated library of BLR variants can be produced by, for example, enzymatically ligating a mixture of synthetic oligonucleotides into gene sequences such that a degenerate set of potential BLR sequences is expressible as individual polypeptides, or alternatively, as a set of larger fusion polypeptides (e.g., for phage display) containing the set of BLR sequences therein. There are a variety of methods which can be used to produce libraries of potential BLR variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential BLR sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In one embodiment, a library of coding sequence fragments can be generated by treating a double stranded PCR fragment of a BLR coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the BLR polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of BLR polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify BLR variants (Arkin and Yourvan (1992) Proc. Natl. Acad. Sci. USA 89:7811-7815; Delgrave et al. (1993) Protein Engineering 6(3):327-331).

In one embodiment, cell based assays can be exploited to analyze a variegated BLR library. For example, a library of expression vectors can be transfected into a cell line which ordinarily synthesizes and secretes BLR. The transfected cells are then cultured such that BLR and a particular mutant BLR are secreted and the effect of expression of the mutant on BLR activity in cell supernatants can be detected, e.g., by any of a number of enzymatic assays. Plasmid DNA can then be recovered from the cells which score for inhibition, or alternatively, potentiation of BLR activity, and the individual clones further characterized.

In addition to BLR polypeptides comprising only naturally-occurring amino acids, BLR peptidomimetics are also provided. Peptide analogs are commonly used in the pharmaceutical industry as non-peptide drugs with properties analogous to those of the template peptide. These types of non-peptide compound are termed “peptide mimetics” or “peptidomimetics” (Fauchere, J. (1986) Adv. Drug Res. 15: 29; Veber and Freidinger (1985) TINS p.392; and Evans et al. (1987) J. Med. Chem 30: 1229, which are incorporated herein by reference) and are usually developed with the aid of computerized molecular modeling.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biological or pharmacological activity), such as BLR, but have one or more peptide linkages optionally replaced by a linkage selected from the group consisting of: —CH2NH—, —CH2S—, —CH2—CH2—, —CH═CH— (cis and trans), —COCH2—, —CH(OH)CH2—, and —CH2SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends Pharm Sci (1980) pp. 463-468 (general review); Hudson, D. et al., Int J Pept Prot Res (1979) 14:177-185 (—CH2NH—, CH2CH2—); Spatola, A. F. et al., Life Sci (1986) 38:1243-1249 (—CH2—S); Hann, M. M., J Chem Soc Perkin Trans I (1982) 307-314 (—CH—CH—, cis and trans); Almquist, R. G. et al., J Med Chem (1980) 23:1392-1398 (—COCH2—); Jennings-White, C. et al., Tetrahedron Lett (1982) 23:2533 (—COCH2—); Szelke, M. et al., European Appln. EP 45665 (1982) CA: 97:39405 (1982)(—CH(OH)CH2—); Holladay, M. W. et al., Tetrahedron Lett (1983) 24:4401-4404 (—C(OH)CH2—); and Hruby, V. J., Life Sci (1982) 31:189-199 (—CH2—S—); each of which is incorporated herein by reference. A particularly preferred non-peptide linkage is —CH2NH—.

Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. derivatization (e.g., labeling) of peptidomimetics should not substantially interfere with the desired biological or pharmacological activity of the peptidomimetic.

Systematic substitution of one or more amino acids of a BLR amino acid sequence with a D-amino acid of the same type (e.g., D-lysine in place of L-lysine) may be used to generate more stable peptides. In addition, constrained peptides comprising a BLR amino acid sequence or a substantially identical sequence variation may be generated by methods known in the art (Rizo and Gierasch (1992) Ann. Rev. Biochem. 61: 387, incorporated herein by reference); for example, by adding internal cysteine residues capable of forming intramolecular disulfide bridges which cyclize the peptide.

The amino acid sequences of BLR polypeptides identified herein will enable those of skill in the art to produce polypeptides corresponding to BLR peptide sequences and sequence variants thereof. Such polypeptides may be produced in prokaryotic or eukaryotic host cells by expression of polynucleotides encoding a BLR peptide sequence, frequently as part of a larger polypeptide. Alternatively, such peptides may be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91: 501; Chaiken I. M. (1981) CRC Crit. Rev. Biochem. 11: 255; Kaiser et al. (1989) Science 243: 187; Merrifield, B. (1986) Science 232: 342; Kent, S. B. H. (1988) Ann. Rev. Biochem. 57: 957; and Offord, R. E. (1980) Semisynthetic Proteins, Wiley Publishing, which are incorporated herein by reference).

Peptides can be produced, typically by direct chemical synthesis, and used e.g., as agonists or antagonists of a BLR molecule, e.g., to modulate binding of a BLR polypeptide and a molecule with which it normally interacts. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, may be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides may be used therapeutically, e.g., to treat infection, either alone or in combination with other agents.

An isolated BLR polypeptide, or a portion or fragment thereof, can also be used as an immunogen to generate antibodies that bind BLR using standard techniques for polyclonal and monoclonal antibody preparation. A full-length BLR polypeptide can be used or, alternatively, the invention provides antigenic peptide fragments of BLR for use as immunogens. The antigenic peptide of BLR preferably comprises at least 8 amino acid residues and encompasses an epitope of BLR such that an antibody raised against the peptide forms a specific immune complex with BLR. More preferably, the antigenic peptide comprises at least 10 amino acid residues, even more preferably at least 15 amino acid residues, even more preferably at least 20 amino acid residues, and most preferably at least 30 amino acid residues.

Alternatively, an antigenic peptide fragment of a BLR polypeptide can be used as the immunogen. An antigenic peptide fragment of a BLR polypeptide typically comprises at least 8 amino acid residues of the amino acid sequence shown in SEQ ID NO: 2 and encompasses an epitope of a BLR polypeptide such that an antibody raised against the peptide forms an immune complex with a BLR molecule. Preferred epitopes encompassed by the antigenic peptide are regions of BLR that are located on the surface of the polypeptide, e.g., hydrophilic regions. In one embodiment, an antibody binds substantially specifically to a BLR molecule. In another embodiment, an antibody binds specifically to a BLR polypeptide.

Preferably, the antigenic peptide comprises at least about 10 amino acid residues, more preferably at least about 15 amino acid residues, even more preferably at least 20 about amino acid residues, and most preferably at least about 30 amino acid residues. Preferred epitopes encompassed by the antigenic peptide are regions of a BLR polypeptide that are located on the surface of the polypeptide, e.g., hydrophilic regions, and that are unique to a BLR polypeptide. In one embodiment such epitopes can be specific for a BLR polypeptides from one species (i.e., an antigenic peptide that spans a region of a BLR polypeptide that is not conserved across species is used as immunogen; such non conserved residues can be determined using an alignment such as that provided herein). A standard hydrophobicity analysis of the polypeptide can be performed to identify hydrophilic regions.

A BLR immunogen typically is used to prepare antibodies by immunizing a suitable subject, (e.g., rabbit, goat, mouse or other mammal) with the immunogen. An appropriate immunogenic preparation can contain, for example, a recombinantly expressed BLR polypeptide or a chemically synthesized BLR peptide. The preparation can further include an adjuvant, such as Freund's complete or incomplete adjuvant, or similar immunostimulatory agent. Immunization of a suitable subject with an immunogenic BLR preparation induces a polyclonal anti-BLR antibody response.

Accordingly, another aspect of the invention pertains to the use of anti-BLR antibodies. Polyclonal anti-BLR antibodies can be prepared as described above by immunizing a suitable subject with a BLR immunogen. The anti-BLR antibody titer in the immunized subject can be monitored over time by standard techniques, such as with an enzyme linked immunosorbent assay (ELISA) using immobilized a BLR polypeptide. If desired, the antibody molecules directed against a BLR polypeptide can be isolated from the mammal (e.g., from the blood) and further purified by well known techniques, such as protein A chromatography to obtain the IgG fraction. At an appropriate time after immunization, e.g., when the anti-BLR antibody titers are highest, antibody-producing cells can be obtained from the subject and used to prepare monoclonal antibodies by standard techniques, such as the hybridoma technique originally described by Kohler and Milstein (1975, Nature 256:495-497) (see also, Brown et al. (1981) J. Immunol 127:539-46; Brown et al. (1980) J Biol Chem 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet., 3:231-36). Briefly, an immortal cell line (typically a myeloma) is fused to lymphocytes (typically splenocytes) from a mammal immunized with a BLR immunogen as described above, and the culture supernatants of the resulting hybridoma cells are screened to identify a hybridoma producing a monoclonal antibody that binds specifically to a BLR polypeptide.

Any of the many well known protocols used for fusing lymphocytes and immortalized cell lines can be applied for the purpose of generating an anti-BLR monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a BLR molecule, e.g., using a standard ELISA assay.

As an alternative to preparing monoclonal antibody-secreting hybridomas, a monoclonal anti-BLR antibody can be identified and isolated by screening a recombinant combinatorial immunoglobulin library (e.g., an antibody phage display library) with a BLR to thereby isolate immunoglobulin library members that bind a BLR polypeptide. Kits for generating and screening phage display libraries are commercially available (e.g., the Pharmacia Recombinant Phage Antibody System, Catalog No. 27-9400-0 1; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. International Publication No. WO 92/18619; Dower et al International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins etal. (1992) J Mol Biol 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; Barbas et al. (1991) PNAS 88:7978-7982; and McCafferty et al. Nature (1990) 348:552-554.

Additionally, recombinant anti-BLR antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in Robinson et al. International Patent Publication PCT/US86/02269; Akira, et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) PNAS 84:3439-3443; Liu et al. (1987) J. Immunol. 139:3521-3526; Sun et al. (1987) PNAS 84:214-218; Nishimura et al. (1987) Canc. Res. 47:999-1005; Wood et al. (1985) Nature 314:446-449; and Shaw et al. (1988) J. Natl Cancer Inst. 80:1553-1559); Morrison, S. L. (1985) Science 229:1202-1207; Oi et al. (1986) BioTechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, humanized antibodies can be made according to standard protocols such as those disclosed in U.S. Pat. No. 5,565,332. In another embodiment, antibody chains or specific binding pair members can be produced by recombination between vectors comprising nucleic acid molecules encoding a fusion of a polypeptide chain of a specific binding pair member and a component of a replicable geneic display package and vectors containing nucleic acid molecules encoding a second polypeptide chain of a single binding pair member using techniques known in the art, e.g., as described in U.S. Pat. Nos. 5,565,332, 5,871,907, or 5,733,743.

An anti-BLR antibody (e.g., monoclonal antibody) can be used to isolate a BLR polypeptide by standard techniques, such as affinity chromatography or immunoprecipitation. Anti-BLR antibodies can facilitate the purification of natural BLR polypeptides from cells and of recombinantly produced BLR polypeptides expressed in host cells. Moreover, an anti-BLR antibody can be used to detect a BLR polypeptide (e.g., in a cellular lysate or cell supernatant). Detection may be facilitated by coupling (i.e., physically linking) the antibody to a detectable substance. Accordingly, in one embodiment, an anti-BLR antibody of the invention is labeled with a detectable substance. Examples of detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹²⁵I, ¹³¹I, ³⁵S or ³H.

III Microbes

Numerous different microbes are suitable for use as sources of BLR nucleic acid molecules or polypeptides, as host cells, and in testing for compounds in the screening assays described herein, e.g., for testing for compounds that modulate the activity and/or expression of a BLR polypeptides. The term “microbe” includes microorganisms having a BLR polypeptide or those that can be engineered to express such a molecule for the purposes of developing a screening assay. Preferably “microbe” refers to unicellular prokaryotic or eukaryotic microbes including bacteria, fungi, or protozoa. In another embodiment, microbes suitable for use in the invention are multicellular, e.g., parasites or fungi. In preferred embodiments, microbes are pathogenic for humans, animals, or plants. In other embodiments, microbes causing environmental problems, e.g., fouling or spoilage or that perform useful functions such as breakdown of plant matter are also preferred. As such, any of these disclosed microbes may be used as intact cells or as sources of materials for cell-free assays as described herein.

In preferred embodiments, microbes for use in the claimed methods are bacteria, either Gram-negative or Gram-positive bacteria. In a preferred embodiment, any bacteria that are shown to become resistant to drugs, preferably antibiotics that affect peptidoglycan systhesis, are appropriate for use in the claimed methods.

In preferred embodiments, microbes are bacteria from the family Enterobacteriaceae. In more preferred embodiments bacteria of a genus selected from the group consisting of: Escherichia, Proteus, Salmonella, Klebsiella, Shigella, Providencia, Enterobacter, Burkholderia, Pseudomonas, Acinetobacter, Aeromonas, Haemophilus, Yersinia, Neisseria, and Erwinia, Rhodopseudomonas, or Burkholderia.

In yet other embodiments, the microbes are Gram-positive bacteria and are from a genus selected from the group consisting of: Lactobacillus, Azorhizobium, Streptomyces, Pediococcus, Photobacterium, Bacillus, Enterococcus, Staphylococcus, Clostridium, Streptococcus, Butyrivibrio, Sphingomonas, Rhodococcus, or Streptomyces.

In yet other embodiments, the microbes are acid fast bacilli, e.g., from the genus Mycobacterium.

In still other embodiments, the microbes are, e.g., selected from a genus selected from the group consisting of: Methanobacterium, Sulfolobus, Archaeoglobil, Rhodobacter, or Sinorhizobium.

In other embodiments, the microbes are fungi. In a preferred embodiment the fungus is from the genus Mucor or Candida, e.g., Mucor racemosus or Candida albicans.

In yet other embodiments, the microbes are protozoa. In a preferred embodiment the microbe is a malaria or cryptosporidium parasite.

IV. Vectors and Host Cells

Preferred BLR polypeptides for use in screening assays are “isolated” or recombinant polypeptides. In one embodiment, BLR polypeptides can be made from isolated nucleic acid molecules. Nucleic acid molecules encoding BLR polypeptides can be used for screening or can be used to produce BLR polypeptides for use in the instant assays. For example, nucleic acid molecules encoding a BLR polypeptide can be isolated (e.g., isolated from the sequences which naturally flank it in the chromosome and from cellular components) and can be used to produce a BLR polypeptide. In one embodiment, a nucleic acid molecule which has been (1) amplified in vitro by, for example, polymerase chain reaction (PCR); (2) recombinantly produced by cloning, or (3) purified, as by cleavage and gel separation; or (4) synthesized by, for example, chemical synthesis can be used to produce BLR polypeptides.

BLR polypeptides can be expressed in a modified form. For example, for secretion of the translated polypeptide into the lumen of the endoplasmic reticulum, into the periplasmic space or into the extracellular environment, appropriate secretion signals may be incorporated into the expressed polypeptide. These signals may be endogenous to the polypeptide or they may be heterologous signals. Polypeptides of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography. Most preferably, high performance liquid chromatography is employed for purification. Well known techniques for refolding proteins may be employed to regenerate active conformation when the polypeptide is denatured during isolation and or purification.

For recombinant production, host cells can be genetically engineered to incorporate nucleic acid molecules of the invention. In one embodiment nucleic acid molecules specifying BLR polypeptides can be placed in a vector. The term “vector” refers to a nucleic acid molecule capable of transporting another nucleic acid molecule to which it has been linked. The term “expression vector” or “expression system” includes any vector, (e.g., a plasmid, cosmid or phage chromosome) containing a gene construct in a form suitable for expression by a cell (e.g., linked to a promoter). In the present specification, “plasmid” and “vector” are used interchangeably, as a plasmid is a commonly used form of vector. Moreover, the invention is intended to include other vectors which serve equivalent functions. A great variety of expression systems can be used to produce the polypeptides of the invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Appropriate vectors-are widely available commercially and it is within the knowledge and discretion of one of ordinary skill in the art to choose a vector which is appropriate for use with a given host cell. The sequences encoding BLR polypeptides can be introduced into a cell on a self-replicating vector or may be introduced into the chromosome of a microbe using homologous recombination or by an insertion element such as a transposon.

The expression system constructs may contain control regions that regulate expression. “Transcriptional regulatory sequence” is a generic term to refer to DNA sequences, such as initiation signals, enhancers, operators, and promoters, which induce or control transcription of polypeptide coding sequences with which they are operably linked. It will also be understood that a recombinant gene encoding a BLR polypeptide can be under the control of transcriptional regulatory sequences which are the same or which are different from those sequences which control transcription of the naturally-occurring BLR gene. Exemplary regulatory sequences are described in Goeddel; Gene Expression Technology: Methods in Enzymology 185, Academic Press, San Diego, Calif. (1990). For instance, any of a wide variety of expression control sequences, that control the expression of a DNA sequence when operatively linked to it, may be used in these vectors to express DNA sequences encoding the BLR polypeptides of this invention. In one embodiment, an expression control sequence is shown in FIG. 1 approximately 0.1 KB upstream from the TnphoA junction. In another embodiment, an expression control sequence is shown in FIG. 1 approximately 1-1.5 kb upstream from the TnphoA junction.

Generally, any system or vector suitable to maintain, propagate or express nucleic acid molecules and/or to express a polypeptide in a host may be used for expression in this regard. The appropriate DNA sequence may be inserted into the expression system by any of a variety of well-known and routine techniques, such as, for example, those set forth in Sambrook et al., Molecular Cloning, A Laboratory Manual, (supra).

Exemplary expression vectors for expression of a gene encoding a BLR polypeptide and capable of replication in a bacterium, e.g., a gram positive, gram negative, or in a cell of a simple eukaryotic fungus such as a Saccharomyces or, Pichia, or in a cell of a eukaryotic organism such as an insect, a bird, a mammal, or a plant, are known in the art. Such vectors may carry functional replication-specifying sequences (replicons) both for a host for expression, for example a Streptomyces, and for a host, for example, E. coli, for genetic manipulations and vector construction. See e.g. U.S. Pat. No. 4,745,056. Suitable vectors for a variety of organisms are described in Ausubel, F. et al., Short Protocols in Molecular Biology, Wiley, N.Y. (1995), and for example, for Pichia, can be obtained from Invitrogen (Carlsbad, Calif.).

Useful expression control sequences, include, for example, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat polypeptide, the promoter for 3-phosphoglycerate kinase or other glycolytic enzymes, the promoters of acid phosphatase, e.g., Pho5, the promoters of the yeast α-mating factors, the polyhedron promoter of the baculovirus system and other sequences known to control the expression of genes of prokaryotic or eukaryotic cells or their viruses, and various combinations thereof. A useful translational enhancer sequence is described in U.S. Pat. No. 4,820,639.

It should be understood that the design of the expression vector may depend on such factors as the choice of the host cell to be transformed and/or the type of polypeptide desired to be expressed. Representative examples of appropriate hosts include bacterial cells, such as gram positive, gram negative cells; fungal cells, such as yeast cells and Aspergillus cells; insect cells such as Drosophila S2 and Spodoplera Sf9 cells; animal cells such as CHO, COS, HeLa, C127, 3T3, BHK, 293 and Bowes melanoma cells; and plant cells.

In preferred embodiments, cells used to express BLR polypeptides for purification or for use in screening assays, e.g., host cells, comprise a mutation which renders any endogenous BLR polypeptide nonfunctional or causes the endogenous polypeptide to not be expressed. In other embodiments, mutations may also be made in other related genes of the host cell, such that there will be no interference from the endogenous host loci.

Introduction of a nucleic acid molecule into the host cell (“transformation”) can be effected by methods described in many standard laboratory manuals, such as Davis et al., Basic Methods In Molecular Biology, (1986) and Sambrook et al., Molecular Cloning: A Laboratory Manual, 2nd Ed., Cold Spring Harbor Laboratory Press, Cold Spring Harbor, N.Y. (1989). Examples include calcium phosphate transfection, DEAE-dextran mediated transfection, transvection, microinjection, cationic lipid-mediated transfection, electroporation, transduction, scrape loading, ballistic introduction and infection.

Purification of BLR polypeptides, e.g., recombinantly expressed polypeptides, can be accomplished using techniques known in the art. For example, if the BLR polypeptide is expressed in a form that is secreted from cells, the medium can be collected. Alternatively, if the BLR polypeptide is expressed in a form that is retained by cells, the host cells can be lysed to release the BLR polypeptide. Such spent medium or cell lysate can be used to concentrate and purify the BLR polypeptide. For example, the medium or lysate can be passed over a column, e.g., a column to which antibodies specific for the BLR polypeptide have been bound. Alternatively, such antibodies can be specific for a non-BLR polypeptide which has been fused to the BLR polypeptide (e.g., as a tag) to facilitate purification of the BLR polypeptide. Other means of purifying BLR polypeptides are known in the art.

V. Uses of BLR Compositions

The BLR modulating agents (e.g., nucleic acid molecules, polypeptides, polypeptide homologues, BLR agonists or antagonists and antibodies described herein) can be used in one or more of the following methods: a) methods of treatment, e.g., treatment of infection, particularly infection with organisms resistant to antibiotics that affect peptidoglycan synthesis; b) screening assays; c) use in vaccines, d) diagnostic assays, and the like. The isolated nucleic acid molecules of the invention can be used, for example, to express BLR polypeptide (e.g., in a host cell in gene therapy applications), to detect BLR mRNA (e.g., in a biological sample) or a genetic alteration in a BLR gene, and to modulate BLR activity, as described further below. The BLR polypeptides can be used to treat infection, (alone or in combination with a second drug, e.g., an antibiotic) or to reduce contamination, e.g., alone or in combination with a non-antibiotic agent. In addition, the BLR polypeptides can be used to screen for naturally occurring BLR binding polypeptides, to screen for drugs or compounds which modulate BLR activity (e.g., are agonists or antagonists of BLR activity), as well as to treat disorders that would benefit from modulation of BLR, e.g., infection with a microbe. Moreover, the anti-BLR antibodies of the invention can be used to detect and isolate BLR polypeptides, regulate the bioavailability of BLR polypeptides, and modulate BLR activity.

A. Methods of Treatment

The subject compositions can be used in treating disorders that would benefit from modulation of a BLR polypeptide activity, e.g., in treating a subject having an infection with a microbe that expresses a BLR polypeptide.

As used herein the term “infection” includes the presence of a microbe in or on a subject which, if its growth and/or virulence were inhibited, would result in a benefit to the subject. As such, the term “infection” in addition to referring to the presence of pathogens also includes normal flora which is not desirable, e.g., on the skin of a burn patient or in the gastrointestinal tract of an immunocompromised patient. As used herein, the term “treating” refers to the administration of a compound to a subject, for prophylactic and/or therapeutic purposes. The term “administration” includes delivery to a subject, e.g., by any appropriate method which serves to deliver the drug to the site of the infection. Administration of the drug can be, e.g., oral, intravenous, or topical (as described in further detail below).

In a preferred embodiment, a microbe which is to be treated is resistant to at least one antibiotic that affects peptidoglycan synthesis.

In one embodiment, a composition of the invention (e.g., a BLR modulating agent) is administered to a subject in combination with additional agents, such as an antibiotic, e.g., an antibiotic that affects peptidoglycan synthesis.

B. Uses in Identifying BLR Agonists and Antagonists

The invention provides a method (also referred to herein as a “screening assay”) to identify those substances which modulate (enhance (agonists) or block (antagonists)) the action of BLR polypeptides or nucleic acid molecules, particularly those compounds that are bacteriostatic and/or bactericidal or prevent the infectious process. The subject screening assays can be used to identify modulators, i.e., candidate or test compounds or agents (e.g., peptides, peptidomimetics, small molecules or other drugs) which modulate BLR polypeptides, i.e., have a stimulatory or inhibitory effect on, for example, BLR polypeptide expression or BLR polypeptide activity. Test compounds may be natural substrates and ligands or may be structural or functional mimetics. See, e.g., Coligan et al., Current Protocols in Immunology 1(2): Chapter 5 (1991).

BLR polypeptide agonists and antagonists can be assayed in a variety of ways. For example, in one embodiment, the invention provides for methods for identifying a compound which modulates the activity or expression of a BLR molecule, e.g., by measuring the ability of the compound to interact with a BLR nucleic acid molecule. Furthermore, the ability of a compound to modulate the binding of a BLR polypeptide or BLR nucleic acid molecule to a molecule to which they normally bind, e.g., a BLR binding polypeptide can be tested.

Compounds for testing in the instant methods can be derived from a variety of different sources and can be known or can be novel. Each of the DNA sequences provided herein may be used in the discovery and development of antibacterial compounds. The encoded proteins, upon expression, can be used as a target for the screening of antibacterial drugs. In another embodiment, antisense nucleic acid molecules or nucleic acid molecules that encode for dominant negative BLR mutants can also be tested in the subject assays.

In one embodiment, libraries of compounds are tested in the instant methods. In another embodiment, known compounds are tested in the instant methods. In another embodiment, compounds among the list of compounds generally regarded as safe (GRAS) by the Environmental Protection Agency are tested in the instant methods.

In one embodiment, a library of compounds can be screened in the subject assays. A recent trend in medicinal chemistry includes the production of mixtures of compounds, referred to as libraries. While the use of libraries of peptides is well established in the art, new techniques have been developed which have allowed the production of mixtures of other compounds, such as benzodiazepines (Bunin et al. 1992. J. Am. Chem. Soc. 114:10987; DeWitt et al. 1993. Proc. Natl. Acad. Sci. USA 90:6909) peptoids (Zuckermann. 1994. J. Med. Chem. 37:2678) oligocarbamates (Cho et al. 1993. Science. 261:1303), and hydantoins (DeWitt et al. supra). Rebek et al. have described an approach for the synthesis of molecular libraries of small organic molecules with a diversity of 10⁴-10⁵ (Carell et al. 1994. Angew. Chem. Int. Ed. Engl. 33:2059; Carell et al. Angew. Chem. Int. Ed. Engl. 1994. 33:2061).

The compounds for screening in the assays of the present invention can be obtained using any of the numerous approaches in combinatorial library methods known in the art, including: biological libraries; spatially addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, non-peptide oligomer or small molecule libraries of compounds (Lam, K. S. Anticancer Drug Des. 1997. 12:145).

Exemplary compounds which can be screened for activity include, but are not limited to, peptides, nucleic acids, carbohydrates, small organic molecules (e.g., polyketides) (Cane et al. 1998. Science 282:63), and natural product extract libraries. In one embodiment, the test compound is a peptide or peptidomimetic. In another, preferred embodiment, the compounds are small, organic non-peptidic compounds.

Other exemplary methods for the synthesis of molecular libraries can be found in the art, for example in: Erb et al. 1994. Proc. Natl. Acad. Sci. USA 91:11422; Horwell et al. 1996 Immunopharmacology 33:68; and in Gallop et al. 1994. J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra). Other types of peptide libraries may also be expressed, see, for example, U.S. Pat. Nos. 5,270,181 and 5,292,646). In still another embodiment, combinatorial polypeptides can be produced from a cDNA library.

BLR polypeptides of the invention increase antibiotic resistance in cells. The ability of a compound to function as a BLR agonist or antagonist can be tested, e.g., by monitoring the effects of the compound on BLR expression or activity. Its efficacy in so doing can be assessed by generating dose response curves from data obtained using various concentrations of the test modulating agent(s). Moreover, a control assay can also be performed to provide a baseline for comparison. As described in more detail below, either whole cell or cell free assay systems can be employed.

1. Whole Cell Assays

In one embodiment of the invention, the subject screening assays can be performed using whole cells. In one embodiment of the invention, the step of determining whether a compound reduces the activity or expression of a BLR polypeptide comprises contacting a cell expressing a BLR polypeptide with a compound and measuring the ability of the compound to modulate the activity or expression of a BLR polypeptide.

In another embodiment, modulators of BLR polypeptide expression are identified in a method wherein a cell is contacted with a candidate compound and the expression of BLR polypeptide mRNA or protein in the cell is determined. The level of expression of BLR polypeptide mRNA or protein in the presence of the candidate compound is compared to the level of expression of BLR polypeptide mRNA or polypeptide in the absence of the candidate compound. The candidate compound can then be identified as a modulator of BLR polypeptide expression based on this comparison. For example, when expression of BLR polypeptide mRNA or protein is greater (e.g., statistically significantly greater) in the presence of the candidate compound than in its absence, the candidate compound is identified as a stimulator of BLR polypeptide mRNA or protein expression. Alternatively, when expression of BLR polypeptide mRNA or protein is less (e.g., statistically significantly less) in the presence of the candidate compound than in its absence, the candidate compound is identified as an inhibitor of BLR mRNA or protein expression. The level of BLR mRNA or protein expression in the cells can be determined by methods described herein for detecting BLR mRNA or protein.

To measure expression of a BLR polypeptide, transcription of a BLR nucleic acid molecule gene can be measured in control cells which have not been treated with the compound and compared with that of test cells which have been treated with the compound. For example, cells which express endogenous BLR polypeptides or which are engineered to express or overexpress recombinant BLR polypeptides can be caused to express or overexpress a recombinant BLR polypeptide in the presence and absence of a test modulating agent of interest, with the assay scoring for modulation in BLR polypeptide responses by the target cell mediated by the test agent. For example, as with the cell-free assays, modulating agents which produce a change, e.g., a statistically significant change in BLR polypeptide-dependent responses (either an increase or decrease) can be identified.

Recombinant expression vectors that can be used for expression of BLR polypeptides are known in the art (see discussions above). In one embodiment, within the expression vector the BLR polypeptide-coding sequences are operatively linked to regulatory sequences that allow for constitutive or inducible expression of BLR polypeptide in the indicator cell(s). Use of a recombinant expression vector that allows for constitutive or inducible expression of BLR polypeptide in a cell is preferred for identification of compounds that enhance or inhibit the activity of BLR polypeptide. In an alternative embodiment, within the expression vector the BLR polypeptide coding sequences are operatively linked to regulatory sequences of the endogenous BLR polypeptide gene (i.e., the promoter regulatory region derived from the endogenous gene). Use of a recombinant expression vector in which BLR polypeptide expression is controlled by the endogenous regulatory sequences is preferred for identification of compounds that enhance or inhibit the transcriptional expression of BLR polypeptide.

In one embodiment, the level of transcription can be determined by measuring the amount of RNA produced by the cell. For example, the RNA can be isolated from cells which express a BLR polypeptide and that have been incubated in the presence and absence of compound. Northern blots using probes specific for the sequences to be detected can then be performed using techniques known in the art. Numerous other, art-recognized techniques can be used. For example, in another embodiment, transcription of specific RNA molecules can be detected using the polymerase chain reaction, for example by making cDNA copies of the RNA transcript to be measured and amplifying and measuring them. In another embodiment, RNAse protection assays, such as S1 nuclease mapping or RNase mapping can be used to detect the level of transcription of a gene. In another embodiment, primer extension can be used.

In yet other embodiments, the ability of a compound to induce a change in transcription or translation of a BLR polypeptide can be accomplished by measuring the amount of BLR polypeptide produced by the cell. For example, western blot analysis can be used to test for BLR. Polypeptides which can be detected include any polypeptides which are produced upon the activation of a BLR responsive promoter, including, for example, both endogenous sequences and reporter gene sequences. In one embodiment, the amount of polypeptide made by a cell can be detected using an antibody against that polypeptide. In other embodiments, the activity of such a polypeptide can be measured.

In one embodiment, other sequences which are regulated by a BLR promoter (e.g., a promoter having sequence identity with a promoter that regulates expression of a BLR gene (e.g., one of the two putative promoters upstream of the BLR gene (at approximately 0.1 kb and 1-1.5 kb upstream of the TnphoA junction of FIG. 1) can be detected. In one embodiment, sequences not normally regulated by a BLR promoter can be assayed by linking them to a promoter that regulates transcription of a BLR polypeptide. For example, sequences can be linked to a BLR promoter, e.g., as illustrated by the promoter approximately 0.1 kb upstream of the TnphoA junction in FIG. 1 (with an upstream limit at about nucleotide 5182 and a transcriptional start site at about nucleotide 5228-5233).

In preferred embodiments, to provide a convenient readout of the transcription from a BLR promoter, such a promoter is linked to a reporter gene, the transcription of which is readily detectable. For example, a bacterial cell, e.g., an E. coli cell, can be transformed as taught in Cohen et al. 1993. J. Bacteriol. 175:7856.

Examples of reporter genes include, but are not limited to, CAT (chloramphenicol acetyl transferase) (Alton and Vapnek (1979), Nature 282: 864-869) luciferase, and other enzyme detection systems, such as beta-galactosidase; firefly luciferase (deWet et al. (1987), Mol. Cell. Biol. 7:725-737); bacterial luciferase (Engebrecht and Silverman (1984), PNAS 1: 4154-4158; Baldwin et al. (1984), Biochemistry 23: 3663-3667); PhoA, alkaline phosphatase (Toh et al. (1989) Eur. J. Biochem. 182: 231-238, Hall et al. (1983) J. Mol. Appl. Gen. 2: 101), human placental secreted alkaline phosphatase (Cullen and Malim (1992) Methods in Enzymol. 216:362-368) and green fluorescent polypeptide (U.S. Pat. No. 5,491,084; WO96/23898).

In yet another embodiment, the ability of a compound to modulate a BLR polypeptide activity, (e,g., to modulate virulence, drug resistance, multidrug resistance, or resistance to an antibiotic that affects peptidoglycan synthesis) can be tested by measuring the ability of the compound to affect the resistance phenotype of the microbe to the drug, e.g. by testing the ability of the microbe to grow in the presence of the drug. For example, the ability of a test compound to modulate the minimal inhibitory concentration (MIC) of the indicator compound can be tested. Such an assay can be performed using a standard methods, e.g., an antibiotic disc assay or an automated growth assay, e.g., using a system such as that commercially available from Viteck. In one embodiment, the method comprises detecting the ability of the compound to modulate growth of a microbe in the presence of one or more antibiotic that affects peptidoglycan synthesis.

In another embodiment, the ability of a test compound to modulate the efflux of a drug from the cell can be tested. In this method, microbes are contacted with a test compound with or without an indicator compound (an indicator compound is one which is normally exported by the cell). The ability of a test compound to inhibit the activity of an efflux pump is demonstrated by determining whether the intracellular concentration of the test compound or the indicator compound (e.g., an antibiotic that affects peptidoglycan synthesis or a dye) is elevated in the presence of the test compound. If the intracellular concentration of the indicator compound is increased in the presence of the test compound as compared to the intracellular concentration in the absence of the test compound, then the test compound can be identified as an inhibitor of an efflux pump. Thus, one can determine whether or not the test compound is an inhibitor of an efflux pump by showing that the test compound affects the ability of an efflux pump present in the microbe to export the indicator compound.

The “intracellular concentration” of an indicator compound includes the concentration of the indicator compound inside the outermost membrane of the microbe. The outermost membrane of the microbe can be, e.g., a cytoplasmic membrane. In the case of Gram-negative bacteria, the relevant “intracellular concentration” is the concentration in the cellular space in which the indicator compound localizes, e.g., the cellular space which contains a target of the indicator compound.

In one embodiment, the method comprises detecting the ability of the compound to reduce resistance in a microbe, e.g., resistance to an antibiotic that affects peptidoglycan synthesis. For example, in one embodiment, the indicator compound comprises a β lactam and the effect of the test compound on the intracellular concentration of β lactam in the microbe is measured. In one embodiment, an increase in the intracellular concentration of an antibiotic can be measured directly, e.g., in an extract of microbial cells. For example, accumulation of a radiolabelled antibiotic can be determined using standard techniques. For instance, microbes can be contacted with a radiolabelled antibiotic as an indicator composition in the presence and absence of a test compound. The concentration of the antibiotic inside the cells can be measured at equilibrium by harvesting cells from the two groups (with and without test compound) and cell associated radioactivity measured with a liquid scintillation counter. In another embodiment, an increase in the intracellular concentration of antibiotic can be measured indirectly, e.g., by a showing that a given concentration of antibiotic when contacted with the microbe is sufficient to inhibit the growth of the microbe in the presence of the test compound, but not in the absence of the test compound.

In another embodiment, measurement of the intracellular concentration of an indicator compound can be facilitated by using an indicator compound which is readily detectable by spectroscopic means. Such a compound may be, for example, a dye, e.g., a basic dye, or a fluorophore. Exemplary indicator compounds include: acridine, ethidium bromode, gentian violet, malachite green, methylene blue, beenzyn viologen, bromothymol blue, toluidine blue, methylene blue, rose bengal, alcyan blue, ruthenium red, fast green, aniline blue, xylene cyanol, bromophenol blue, coomassie blue, bormocresol purple, bromocresol green, trypan blue, and phenol red.

In such an assay, the effect of the test compound on the ability of the cell to export the indicator compound can be measured spectroscopically. For example, the intracellular concentration of the dye or fluorophore can be determined indirectly, by determining the concentration of the indicator compound in the suspension medium or by determining the concentration of the indicator compound in the cells. This can be done, e.g., by extracting the indicator compound from the cells or by visual inspection of the cells themselves.

In another embodiment, the presence of an indicator compound in a microbe can be detected using a reporter gene which is sensitive to the presence of the indicator compound. Exemplary reporter genes are known in the art. For example, a reporter gene can provide a colorometric read out or an enzymatic read out of the presence of an indicator compound. In yet another embodiment, a reporter gene whose expression is inducible by the presence of a drug in a microbe can be used. For example, a microbe can be grown in the presence of a drug with and without a putative test compound. In cells in which the efflux pump is inhibited, the concentration of the drug will be increased and the reporter gene construct will be expressed. By this method, efflux pump inhibitors are identified by their ability to inhibit the export rate of the drug and, thus, to induce reporter gene expression.

In another embodiment, a primary screening assay is used in which an indicator compound which does not comprise an antibiotic is employed. In one embodiment, upon the identification of a test compound that increases the intracellular concentration of the test compound, a secondary screening assay is performed in which the effect of the same test compound on susceptibility to the drug of interest, e.g., resistance to an antibiotic that affects peptidoglycan synthesis, is measured.

In yet another embodiment, the ability of a compound to modulate the binding of a BLR polypeptide to a BLR binding polypeptide can be determined. BLR binding polypeptides can be identified using techniques which are known in the art. For example, in the case of binding polypeptides that interact with BLR polypeptides, interaction trap assays or two hybrid screening assays can be used.

BLR binding polypeptides can be identified e.g., e.g., by using a BLR polypeptides or portions thereof of the invention as a “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO94/10300), to identify other proteins, which bind to or interact with BLR polypeptides (“BLR-binding polypeptides”) and are involved in BLR activity. Such BLR family-binding polypeptides are also likely to be involved in the propagation of signals by the BLR polypeptides or to associate with BLR polypeptides and enhance or inhibit their activity.

The two-hybrid system is based on the modular nature of most transcription factors, which consist of separable DNA-binding and activation domains. Briefly, the assay utilizes two different DNA constructs. In one construct, the gene that codes for a BLR polypeptide is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a BLR polypeptide-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the polypeptide which interacts with the BLR polypeptide.

BLR binding polypeptides may also be identified in other ways. For example, a library of molecules can be tested for the presence of BLR binding polypeptides. In one embodiment, the library of molecules can be tested by expressing them in an expression vector, e.g., a bacteriophage. Bacteriophage can be made to display on their surface a plurality of polypeptide sequences, each polypeptide sequence being encoded by a nucleic acid molecule contained within the bacteriophage. The phage expressing these candidate BLR binding polypeptides can be tested for the ability to bind an immobilized BLR polypeptide, to obtain those polypeptides having affinity for the BLR polypeptide. For example, the method can comprise: contacting the immobilized BLR polypeptide with a sample of the library of bacteriophage so that the BLR polypeptide can interact with the different polypeptide sequences and bind those having affinity for the BLR polypeptide to form a set of complexes consisting of immobilized BLR polypeptide and bound bacteriophage. The complexes which have not formed a complex can be separated. The complexes of BLR polypeptide and bound bacteriophage can be contacted with an agent that dissociates the bound bacteriophage from the complexes; and the dissociated bacteriophage can be isolated and the sequence of the nucleic acid molecule encoding the displayed polypeptide obtained, so that amino acid sequences of displayed polypeptides with affinity for BLR polypeptides are obtained.

In the case of BLR nucleic acid molecules, BLR binding polypeptides can be identified, e.g., by contacting a BLR nucleotide sequence with candidate BLR binding polypeptides (e.g., in the form of microbial extract) under conditions which allow interaction of components of the extract with the BLR nucleotide sequence. The ability of the BLR nucleotide sequence to interact with the components can then be measured to thereby identify a polypeptide that binds to a BLR nucleotide sequence.

2. Cell-Free Assays

The subject screening methods can involve cell-free assays, e.g., using high-throughput techniques. For example, to screen for BLR agonists or antagonists, a synthetic reaction mix comprising a BLR molecule and a labeled substrate or ligand of such polypeptide is incubated in the absence or the presence of a candidate molecule that may be an agonist or antagonist. In one embodiment, the reaction mix can further comprise a cellular compartment, such as a membrane, cell envelope or cell wall, or a combination thereof. The ability of the test compound to agonize or antagonize the BLR polypeptide is reflected in decreased binding of the BLR polypeptide to a BLR binding polypeptide or in a decrease in BLR polypeptide activity.

In many drug screening programs which test libraries of modulating agents and natural extracts, high throughput assays are desirable in order to maximize the number of modulating agents surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system.

In one embodiment, the ability of a compound to modulate the activity of a BLR polypeptide is accomplished using isolated BLR polypeptides or BLR nucleic acid molecule molecule in a cell-free system. In such an assay, the step of measuring the ability of a compound to modulate the activity of the BLR polypeptide is accomplished, for example, by measuring direct binding of the compound to a BLR polypeptide or BLR nucleic acid molecule or the ability of the compound to alter the ability of the BLR polypeptide to bind to a BLR binding polypeptide to which the BLR polypeptide normally binds.

In yet another embodiment, an assay of the present invention is a cell-free assay in which a BLR polypeptide or portion thereof is contacted with a test compound and the ability of the test compound to bind to the BLR polypeptide or biologically active portion thereof is determined. Determining the ability of the test compound to modulate the activity of a BLR polypeptide can be accomplished, for example, by determining the ability of the BLR polypeptide to bind to a BLR target molecule by one of the methods described above for determining direct binding. Determining the ability of the BLR polypeptide to bind to a BLR target molecule can also be accomplished using a technology such as real-time Biomolecular Interaction Analysis (BIA). Sjolander, S. and Urbaniczky, C. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In yet another embodiment, the cell-free assay involves contacting a BLR polypeptide or biologically active portion thereof with a known compound which binds the BLR polypeptide to form an assay mixture, contacting the assay mixture with a test compound, and determining the ability of the test compound to interact with the BLR polypeptide, wherein determining the ability of the test compound to interact with the BLR polypeptide comprises determining the ability of the BLR polypeptide to preferentially bind to or modulate the activity of a BLR target molecule.

The cell-free assays of the present invention are amenable to use of both soluble and/or membrane-bound forms of proteins (e.g., BLR polypeptides or BLR binding polypeptides). In the case of cell-free assays in which a membrane-bound form a polypeptide is used it may be desirable to utilize a solubilizing agent such that the membrane-bound form of the polypeptide is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

For example, compounds can be tested for their ability to directly bind to a BLR nucleic acid molecule or a BLR polypeptide or portion thereof, e.g., by using labeled compounds, e.g., radioactively labeled compounds. For example, a BLR polypeptide sequence can be expressed by a bacteriophage. In this embodiment, phage which display the BLR polypeptide would then be contacted with a compound so that the polypeptide can interact with and potentially form a complex with the compound. Phage which have formed complexes with compounds can then be separated from those which have not. The complex of the polypeptide and compound can then be contacted with an agent that dissociates the bacteriophage from the compound. Any compounds that bound to the polypeptide can then be isolated and identified.

In another embodiment, the ability of a compound to bind to a BLR nucleic acid molecule can be measured. For example, gel shift assays or restriction enzyme protection assays can be used. Gel shift assays separate polypeptide-DNA complexes from free DNA by non-denaturing polyacrylamide gel electrophoresis. In such an experiment, the level of binding of a compound to DNA can be determined and compared to that in the absence of compound. Compounds which change the level of this binding are selected in the screen as modulating the activity of a BLR polypeptide.

Other methods of assaying the ability of proteins to bind to DNA, e.g., DNA footprinting, and nuclease protection are also well known in the art and can be used to test the ability of a compound to bind to a BLR nucleotide sequence.

In another embodiment, the invention provides a method for identifying compounds that modulate antibiotic resistance by assaying for test compounds that bind to BLR nucleic acid molecules and interfere, e.g., with gene transcription.

In another embodiment, a BLR nucleic acid molecule and a BLR binding polypeptide that normally binds to that nucleotide sequence are contacted with a test compound to identify compounds that block the interaction of a BLR nucleic acid molecule and a BLR binding polypeptide. For example, in one embodiment, the BLR nucleotide sequence and/or the BLR binding polypeptide are contacted under conditions which allow interaction of the compound with at least one of the BLR nucleic acid molecule and the BLR binding polypeptide. The ability of the compound to modulate the interaction of the BLR nucleotide sequence with the BLR binding polypeptide is indicative of its ability to modulate a BLR polypeptide activity.

Determining the ability of the BLR polypeptide to bind to or interact with a BLR binding polypeptide can be accomplished, e.g., by direct binding or by determining the effect of a compound on BLR polypeptide activity. In a direct binding assay, the BLR polypeptide could be coupled with a radioisotope or enzymatic label such that binding of the BLR polypeptide to a BLR polypeptide target molecule can be determined by detecting the labeled BLR polypeptide in a complex. For example BLR polypeptides can be labeled with ¹²⁵I, ³⁵S, ¹⁴C, or ³H, either directly or indirectly, and the radioisotope detected by direct counting of radioemmission or by scintillation counting. Alternatively, BLR polypeptide molecules can be enzymatically labeled with, for example, horseradish peroxidase, alkaline phosphatase, or luciferase, and the enzymatic label detected by determination of conversion of an appropriate substrate to product.

Typically, it will be desirable to immobilize either BLR polypeptide, a BLR binding polypeptide or a compound to facilitate separation of complexes from uncomplexed forms, as well as to accommodate automation of the assay. Binding of BLR polypeptide to an upstream or downstream binding polypeptide, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the polypeptide to be bound to a matrix. For example, glutathione-S-transferase/BLR polypeptide (GST/BLR polypeptide) fusion proteins can be adsorbed onto glutathione sepharose beads (Sigma Chemical, St. Louis, Mo.) or glutathione derivatized microtitre plates, which are then combined with the cell lysates, e.g. an ³⁵S-labeled, and the test modulating agent, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of BLR polypeptide-binding polypeptide found in the bead fraction quantitated from the gel using standard electrophoretic techniques.

Other techniques for immobilizing proteins on matrices are also available for use in the subject assay. For instance, either a BLR polypeptide or polypeptide to which it binds can be immobilized utilizing conjugation of biotin and streptavidin. For instance, biotinylated BLR polypeptide molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with BLR polypeptide but which do not interfere with binding of upstream or downstream elements can be derivatized to the wells of the plate, and BLR polypeptide trapped in the wells by antibody conjugation. As above, preparations of a BLR polypeptide-binding polypeptide and a test modulating agent are incubated in the BLR polypeptide-presenting wells of the plate, and the amount of complex trapped in the well can be quantitated. Exemplary methods for detecting such complexes, in addition to those described above for the GST-immobilized complexes, include immunodetection of complexes using antibodies reactive with the BLR binding polypeptide, or which are reactive with BLR polypeptide and compete with the binding polypeptide; as well as enzyme-linked assays which rely on detecting an enzymatic activity associated with the binding polypeptide, either intrinsic or extrinsic activity. In the instance of the latter, the enzyme can be chemically conjugated or provided as a fusion protein with the BLR binding polypeptide. To illustrate, the BLR polypeptide can be chemically cross-linked or genetically fused with horseradish peroxidase, and the amount of protein trapped in the complex can be assessed with a chromogenic substrate of the enzyme, e.g. 3,3′-diamino-benzadine terahydrochloride or 4-chloro-1-napthol. Likewise, a fusion protein comprising the protein and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the polypeptide, such as anti-BLR polypeptide antibodies, can be used. Alternatively, the polypeptide to be detected in the complex can be “epitope tagged” in the form of a fusion protein which includes, in addition to the BLR polypeptide sequence, a second polypeptide for which antibodies are readily available (e.g. from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharamacia, N.J.).

It is also within the scope of this invention to determine the ability of a compound to modulate the interaction between BLR polypeptide and its target molecule, without the labeling of any of the interactants. For example, a microphysiometer can be used to detect the interaction of BLR polypeptide with its target molecule without the labeling of either BLR polypeptide or the target molecule. McConnell, H. M. et al. (1992) Science 257:1906-1912. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

This invention further pertains to novel agents identified by the above-described screening assays. Accordingly, it is within the scope of this invention to further use an agent identified as described herein in methods of reducing drug resistance in microbes, e.g., in vivo or ex vivo. For example, an agent identified as described herein (e.g., a BLR modulating agent, an antisense BLR nucleic acid molecule, an antagonist, a BLR family-specific antibody, or a BLR-binding partner) can be used in an animal model to determine the efficacy, toxicity, or side effects of treatment with such an agent. Alternatively, an agent identified as described herein can be used in an animal model to determine the mechanism of action of such an agent. Additionally, such agents can be used in methods of treatment (in vivo or ex vivo) or in methods of reducing resistance to drugs in the environment. Furthermore, this invention pertains to uses of novel agents identified by the above-described screening assays for treatments as described herein.

The modulating agents of the invention may be employed, for instance, to inhibit and treat disease, such as, infections. Preferably, such BLR modulating agents are used to treat infection with organisms resistant to an antibiotic that affects peptidoglycan synthesis.

C. Vaccines

Another aspect of the invention relates to a method for inducing an immunological response in an individual, particularly a mammal, comprising inoculating the individual with a BLR modulating agent, e.g., a BLR polypeptide or a fragment or variant thereof, adequate to produce an immune response (e.g., an antibody and/or T cell immune response) to ameliorate or prevent infection with a microbe comprising a BLR polypeptide. The invention also relates to a method of inducing immunological response in an individual which comprises delivering to such individual a nucleic acid vector to direct expression of a BLR molecule, or a fragment or a variant thereof, for expressing a BLR molecule, or a fragment or a variant thereof in vivo in order to induce an immunological response, such as, to produce antibody and/or T cell immune response, including, for example, cytokine-producing T cells or cytotoxic T cells, to ameliorate an ongoing infection or to prevent infection. One way of administering the gene is by accelerating it into the desired cells as a coating on particles or otherwise. Such nucleic acid vector may comprise, e.g., DNA, RNA, a modified nucleic acid, or a DNA/RNA hybrid.

A further aspect of the invention relates to an immunological composition which, when introduced into an individual, induces an immunological response. Such a composition can comprise, e.g., an isolated BLR polypeptide or a BLR nucleic acid molecule. The immunologic composition may be used therapeutically or prophylactically and may be dominated by either a humoral response or a cellular immune response.

In one embodiment, a BLR polypeptide or a fragment thereof may be fused with a second polypeptide, which may not by itself produce antibodies, but is capable of stabilizing the first polypeptide and enhancing immunogenic and protective properties. Thus fused recombinant polypeptide, preferably further comprises an antigenic co-protein, such as lipoprotein D from Hemophilus influenzae, Glutathione-S-transferase (GST) or beta-galactosidase, relatively large second proteins which solubilize the polypeptide and facilitate production and purification of a BLR molecule to which they are fused. Moreover, the second polypeptide may act as an adjuvant in the sense of providing a generalized stimulation of the immune system. The second polypeptide may be attached to either the amino or carboxy terminus of the BLR polypeptide.

The use of a nucleic acid molecule of the invention in genetic immunization will preferably employ a suitable delivery method such as direct injection of plasmid DNA into muscles (Wolff et al., Hum Mol Genet 1992, 1:363, Manthorpe et al., Hum. Gene Ther. 1963:4, 419), delivery of DNA complexed with specific polypeptide carriers (Wu et al., J Biol Chem. 1989: 264,16985), coprecipitation of DNA with calcium phosphate (Benvenisty & Reshef, PNAS USA, 1986:83,9551), encapsulation of DNA in various forms of liposomes (Kaneda et al., Science 1989:243,375), particle bombardment (Tang et al., Nature 1992, 356:152, Eisenbraun et al., DNA Cell Biol 1993, 12:791) and in vivo infection using cloned retroviral vectors (Seeger et al., PNAS USA 1984:81,5849).

In one embodiment, immunostimulatory DNA sequences, such as those described in Sato, Y. et al. Science 273: 352 (1996) can be used in connection with the instant invention.

In one embodiment, a vaccine formulation comprises an immunogenic recombinant polypeptide of the invention together with a suitable carrier. Preferably, such vaccines are administered parenterally, including, for example, administration that is subcutaneous, intramuscular, intravenous, or intradermal. Formulations suitable for parenteral administration include aqueous and non-aqueous sterile injection solutions which may contain anti-oxidants, buffers, bacteriostats and solutes which render the formulation isotonic with the bodily fluid, preferably the blood, of the individual; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems, alum, or other systems known in the art. The dosage will depend on the specific activity of the vaccine and (possibly) on the status of the patient and can be readily determined by routine experimentation.

VI. Compositions Comprising BLR Modulating Agents

The compositions of the invention can comprise one or more pharmaceutically acceptable carriers (additives) and/or diluents. A composition can also include a second antimicrobial agent, e.g., an antimicrobial compound, preferably an antibiotic, e.g., an antibiotic that affects peptidoglycan synthesis.

As described in detail below, the compositions can be formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream, foam, or suppository; or (5) aerosol, for example, as an aqueous aerosol, liposomal preparation or solid particles containing the compound.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the antimicrobial agents or compounds of the invention from one organ, or portion of the body, to another organ, or portion of the body without affecting its biological effect. Each carrier should be “acceptable” in the sense of being compatible with the other ingredients of the composition and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. Proper fluidity can be maintained, for example, by the use of coating materials, such as lecithin, by the maintenance of the required particle size in the case of dispersions, and by the use of surfactants.

These compositions may also contain additional agents, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Pharmaceutical compositions of the present invention may be administered to epithelial surfaces of the body orally, parenterally, topically, rectally, nasally, intravaginally, intracisternally. They are of course given by forms suitable for each administration route. For example, they are administered in tablets or capsule form, by injection, inhalation, eye lotion, ointment, etc., administration by injection, infusion or inhalation; topical by lotion or ointment; and rectal or vaginal suppositories.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a sucrose octasulfate and/or an antibacterial or a contraceptive agent, drug or other material other than directly into the central nervous system, such that it enters the subject's system and, thus, is subject to metabolism and other like processes, for example, subcutaneous administration.

In some methods, the compositions of the invention can be topically administered to any epithelial surface. An “epithelial surface” according to this invention is defined as an area of tissue that covers external surfaces of a body, or which and lines hollow structures including, but not limited to, cutaneous and mucosal surfaces. Such epithelial surfaces include oral, pharyngeal, esophageal, pulmonary, ocular, aural, nasal, buccal, lingual, vaginal, cervical, genitourinary, alimentary, and anorectal surfaces.

Compositions can be formulated in a variety of conventional forms employed for topical administration. These include, for example, semi-solid and liquid dosage forms, such as liquid solutions or suspensions, suppositories, douches, enemas, gels, creams, emulsions, lotions, slurries, powders, sprays, lipsticks, foams, pastes, toothpastes, ointments, salves, balms, douches, drops, troches, chewing gums, lozenges, mouthwashes, rinses.

Conventionally used carriers for topical applications include pectin, gelatin and derivatives thereof, polylactic acid or polyglycolic acid polymers or copolymers thereof, cellulose derivatives such as methyl cellulose, carboxymethyl cellulose, or oxidized cellulose, guar gum, acacia gum, karaya gum, tragacanth gum, bentonite, agar, carbomer, bladderwrack, ceratonia, dextran and derivatives thereof, ghatti gum, hectorite, ispaghula husk, polyvinypyrrolidone, silica and derivatives thereof, xanthan gum, kaolin, talc, starch and derivatives thereof, paraffin, water, vegetable and animal oils, polyethylene, polyethylene oxide, polyethylene glycol, polypropylene glycol, glycerol, ethanol, propanol, propylene glycol (glycols, alcohols), fixed oils, sodium, potassium, aluminum, magnesium or calcium salts (such as chloride, carbonate, bicarbonate, citrate, gluconate, lactate, acetate, gluceptate or tartrate).

Such compositions can be particularly useful, for example, for treatment or prevention of an unwanted infections e.g., of the oral cavity, including cold sores, infections of eye, the skin, or the lower intestinal tract. Standard composition strategies for topical agents can be applied to the antimicrobial compounds, or pharmaceutically acceptable salts thereof in order to enhance the persistence and residence time of the drug, and to improve the prophylactic efficacy achieved.

For topical application to be used in the lower intestinal tract or vaginally, a rectal suppository, a suitable enema, a gel, an ointment, a solution, a suspension or an insert can be used. Topical transdermal patches may also be used. Transdermal patches have the added advantage of providing controlled delivery of the compositions of the invention to the body. Such dosage forms can be made by dissolving or dispersing the agent in the proper medium.

Compositions of the invention can be administered in the form of suppositories for rectal or vaginal administration. These can be prepared by mixing the agent with a suitable non-irritating carrier which is solid at room temperature but liquid at rectal temperature and therefore will melt in the rectum or vagina to release the drug. Such materials include cocoa butter, beeswax, polyethylene glycols, a suppository wax or a salicylate.

Compositions which are suitable for vaginal administration also include pessaries, tampons, creams, gels, pastes, foams, films, or spray compositions containing such carriers as are known in the art to be appropriate. The carrier employed in the sucrose octasulfate/contraceptive agent should be compatible with vaginal administration and/or coating of contraceptive devices. Combinations can be in solid, semi-solid and liquid dosage forms, such as diaphragm, jelly, douches, foams, films, ointments, creams, balms, gels, salves, pastes, slurries, vaginal suppositories, sexual lubricants, and coatings for devices, such as condoms, contraceptive sponges, cervical caps and diaphragms.

For ophthalmic applications, the pharmaceutical compositions can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the compositions can be formulated in an ointment such as petrolium. Exemplary ophthalmic compositions include eye ointments, powders, solutions and the like.

Powders and sprays can contain, in addition to sucrose octasulfate and/or antibiotic or contraceptive agent(s), carriers such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Ordinarily, an aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Compositions of the invention can also be orally administered in any orally-acceptable dosage form including, but not limited to, capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of sucrose octasulfate and/or antibiotic or contraceptive agent(s) as an active ingredient. A compound may also be administered as a bolus, electuary or paste. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Tablets, and other solid dosage forms, such as dragees, capsules, pills and granules, may be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the antimicrobial agent(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Sterile injectable forms of the compositions of this invention can be aqueous or oleaginous suspensions. These suspensions may be formulated according to techniques known in the art using suitable dispersing or wetting agents and suspending agents. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

The sterile injectable preparation may also be a sterile injectable solution or suspension in a nontoxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono-or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

In the case of modulators of the activity and/or expression of BLR molecules which are nucleic acid molecules, the optimal course of administration of the oligomers may vary depending upon the desired result or on the subject to be treated. As used in this context “administration” refers to contacting cells with oligomers, e.g., in vivo or ex vivo. The dosage of nucleic molecule may be adjusted to optimally regulate expression of a protein translated from a target mRNA, e.g., as measured by a readout of RNA stability or by a therapeutic response, without undue experimentation. For example, expression of the protein encoded by the nucleic acid can be measured to determine whether or dosage regimen needs to be adjusted accordingly. In addition, an increase or decrease in RNA and/or protein levels in a cell or produced by a cell can be measured using any art recognized technique. By determining whether transcription has been decreased, the effectiveness of the molecule can be determined.

As used herein, “pharmaceutically acceptable carrier” includes appropriate solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredient, it can be used in the therapeutic compositions. Supplementary active ingredients can also be incorporated into the compositions.

Compositions may be incorporated into liposomes or liposomes modified with polyethylene glycol or admixed with cationic lipids for parenteral administration. Incorporation of additional substances into the liposome, for example, antibodies reactive against membrane proteins found on specific target microbes, can help target the molecule to specific cell types.

Moreover, the present invention provides for administering the subject compositions with an osmotic pump providing continuous infusion of the compositions, for example, as described in Rataiczak et al. (1992 Proc. Natl. Acad Sci. USA 89:11823-11827). Such osmotic pumps are commercially available, e.g., from Alzet Inc. (Palo Alto, Calif.). Topical administration and parenteral administration in a cationic lipid carrier are preferred.

With respect to in vivo applications, the formulations of the present invention can be administered to a patient in a variety of forms adapted to the chosen route of administration, namely, parenterally, orally, or intraperitoneally. Parenteral administration, which is preferred, includes administration by the following routes: intravenous; intramuscular; interstitially; intraarterially; subcutaneous; intra ocular; intrasynovial; trans epithelial, including transdermal; pulmonary via inhalation; ophthalmic; sublingual and buccal; topically, including ophthalmic; dermal; ocular; rectal; and nasal inhalation via insufflation. Intravenous administration is preferred among the routes of parenteral administration.

Pharmaceutical preparations for parenteral administration include aqueous solutions of the active compounds in water-soluble or water-dispersible form. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of the suspension include, for example, sodium carboxymethyl cellulose, sorbitol, and/or dextran, optionally, the suspension may also contain stabilizers.

Drug delivery vehicles can be chosen e.g., for in vitro, for systemic, or for topical administration. These vehicles can be designed to serve as a slow release reservoir or to deliver their contents directly to the target cell. An advantage of using some direct delivery drug vehicles is that multiple molecules are delivered per uptake. Such vehicles have been shown to increase the circulation half-life of drugs that would otherwise be rapidly cleared from the blood stream. Some examples of such specialized drug delivery vehicles which fall into this category are liposomes, hydrogels, cyclodextrins, biodegradable nanocapsules, and bioadhesive microspheres.

The subject compositions may be incorporated into liposomes or liposomes modified with polyethylene glycol or admixed with cationic lipids for parenteral administration. Incorporation of additional substances into the liposome, for example, antibodies reactive against membrane proteins found on specific target microbes, can help target the compositions to specific cell types.

Moreover, the present invention provides for administering the subject compositions with an osmotic pump providing continuous infusion of nucleic acid molecules, for example, as described in Rataiczak et al. (1992 Proc. Natl. Acad. Sci. USA 89:11823-11827). Such osmotic pumps are commercially available, e.g., from Alzet Inc. (Palo Alto, Calif.). Topical administration and parenteral administration in a cationic lipid carrier are preferred.

The described compositions may be administered systemically to a subject. Systemic absorption refers to the entry of drugs into the blood stream followed by distribution throughout the entire body. Administration routes which lead to systemic absorption include: intravenous, subcutaneous, intraperitoneal, and intranasal. Each of these administration routes delivers the compositions to accessible diseased cells. Following subcutaneous administration, the therapeutic agent drains into local lymph nodes and proceeds through the lymphatic network into the circulation. The rate of entry into the circulation has been shown to be a function of molecular weight or size. The use of a liposome or other drug carrier localizes the compositions at the lymph node. The nucleic acid molecule can be modified to diffuse into the cell, or the liposome can directly participate in the delivery of the composition into the cell.

For prophylactic applications, the pharmaceutical composition of the invention can be applied prior to physical contact between a patient and a microbe. The timing of application prior to physical contact can be optimized to maximize the prophylactic effectiveness of the compound. The timing of application will vary depending on the mode of administration, the epithelial surface to which it is applied, the surface area, doses, the stability and effectiveness of composition under the pH of the epithelial surface, the frequency of application, e.g., single application or multiple applications. Preferably, the timing of application can be determined such that a single application of composition is sufficient. One skilled in the art will be able to determine the most appropriate time interval required to maximize prophylactic effectiveness of the compound.

One of ordinary skill in the art can determine and prescribe the effective amount of the pharmaceutical composition required. For example, one could start doses at levels lower than that required in order to achieve the desired therapeutic effect and gradually increase the dosage until the desired effect is achieved. In general, a suitable daily dose of a composition of the invention will be that amount of the composition which is the lowest dose effective to produce a therapeutic effect. Such an effective dose will generally depend upon the factors described above. It is preferred that administration be intravenous, intracoronary, intramuscular, intraperitoneal, or subcutaneous.

The practice of the present invention will employ, unless otherwise indicated, conventional techniques of cell biology, cell culture, molecular biology, genetics, microbiology, recombinant DNA, and immunology, which are within the skill of the art. Such techniques are explained fully in the literature. See, for example, Genetics; Molecular Cloning A Laboratory Manual, 2nd Ed., ed. by Sambrook, J. et al. (Cold Spring Harbor Laboratory Press (1989)); Short Protocols in Molecular Biology, 3rd Ed., ed. by Ausubel, F. et al. (Wiley, N.Y. (1995)); DNA Cloning, Volumes I and II (D. N. Glover ed., 1985); Oligonucleotide Synthesis (M. J. Gait ed. (1984)); Mullis et al. U.S. Pat. No: 4,683,195; Nucleic Acid Hybridization (B. D. Hames & S. J. Higgins eds. (1984)); the treatise, Methods In Enzymology (Academic Press, Inc., N.Y); Immunochemical Methods In Cell And Molecular Biology (Mayer and Walker, eds., Academic Press, London (1987)); Handbook Of Experimental Immunology, Volumes I-IV (D. M. Weir and C. C. Blackwell, eds. (1986)); and Miller, J. Experiments in Molecular Genetics (Cold Spring Harbor Press, Cold Spring Harbor, N.Y. (1972)).

The contents of all references, pending patent applications and published patents, cited throughout this application are hereby expressly incorporated by reference. Each reference disclosed herein is incorporated by reference herein in its entirety. Any patent application to which this application claims priority is also incorporated by reference herein in its entirety.

The invention is further illustrated by the following examples, which should not be construed as further limiting.

### EXAMPLES

### Example 1

Isolation of a 358 Base Pairs Sequence that Increases Susceptibility to Antibiotics.

Donor E. coli K 12 strain S17-1 λpir (Tp^{r}Sm^{r}recA, thi, pro, hsdR⁻M⁺RP4;2-Tc:Mu:KmTn7,λpir; George and Levy. 1983. J Bacteriol 1983 Aug; 155 (2): 531-48) containing a conjugatable transposon-delivery plasmid pUT/Km (able to replicate only in the donor) which bore mini-Tn5phoA (de Lorenzo, V. & Timmis, K. N. Meth. Enzymol. 235, 386-404 (1994)) was mated with recipient ASS111rif and plated on kanamycin/rifamycin. Transconjugant RW583 had both PhoA activity and a 2-4 fold enhanced susceptibility to a wide spectrum of antibiotics an antibiotic that affects peptidoglycan synthesis including cephalosporins and imipenem (Table 1) as well as cycloserine and bacitracin, while susceptibility to tetracycline, chloramphenicol, nalidixic acid, norfloxacin, gentamicin, fosfomycin or valinomycin was not affected. The insertion was transduced (Provence, D. L. & Curtiss, R. I. in Methods for General and Molecular Bacteriology (ed. Gerhardt, P., Murry, R. G. E., Wood, W. A. & Kreig, N. R.) 317-347 (American Society for Microbiology, Washington D.C. 1994)) by bacteriophage P1 into other E. coli K12 strains, where it had the same effect on susceptibility (Table 1).

A 6.5 kb BamHI chromosomal fragment from RW583 containing the phoA and kan genes from TnphoA was identified by cloning into the BamHI site of pBR322 and selection upon kanamycin. The sequence of the clone revealed that the insertion was at nucleotide 1702674 of the genome (min 36.6) in a hypothetical intergenic region (see GenBank accession numbers AE000258 and D90807) of 602 base pairs between two divergent ORFs. By the annotation of AE000258, the divergent ORFs are b1624 (ORF359, putative oxidoreductase, on the—strand) and b1625 (ORF71, putative histone-like negative regulator) (FIG. 1). The intergenic locus was named “blr” (beta lactam resistance). Other potential upstream translational starts that are also in frame with the phoA of TnphoA are shown in small bold font. The 21 residue transmembrane domain predicted for BLR by TopPred 218 is in small bold font with underlining. The transcriptional start is indicated by the “=” of “+mRNA”. Regions corresponding to primers F2, R1, R2, and R3 are underlined or overlined. Amber mutations (TAG) are labeled above the sequence. Additional sequences carrying restriction enzyme sites were added to the 5′ ends of certain of the primers, creating oligonucleotides F2a, with addition ′TTTAAAGCTT (SEQ ID NO: 3) (DraI, HindIII); R1A, with addition 5′ ACTAGTACTGCAG (SEQ ID NO: 4) (PstI, ScaI) and R3A, with addition 5′CGGGAAGCTT3′ (SEQ ID NO: 5) (HindIII). The putative start codon for ORF71, reading rightward, is out of view at nucleotide 5670. Putative promoters annotated in GenBank AE000258 are noted by a “P” followed by dots above the sequence. The sequence of the blr region as cloned in all plasmids except pRW23A has a T to C PCR error at nucleotide 5449, which does not affect the blr gene.

In the blr locus, there are 11 ORFs which are interrupted by, and in frame with, phoA. Al stop with the TAA codon at nucleotide 5395-7 (FIG. 1); the largest has only 66 amino acids. A 358 base pair region encompassing this ORF and adjacent sequences was synthesized as a PCR fragment using oligonucleotides F2a and R1A (supra) and cloned into pUC19 using PstI;HindIII. To permit tests of beta-lactam susceptibility, the ScaI/AatII fragment in the ampicillin resistance gene of the resulting pUC 19 derivative was replaced by a SmaI/AatII spectinomycin resistance cassette from pFW12 (Podbielski, A., Spellerberg, B., Woischnik, M., Pohl, B. & Lutticken, R. Novel series of plasmid vectors for gene inactivation and expression analysis in group A streptococci (GAS). Gene 177: 137-147 (1996)). In the resulting plasmid, pRW23C, blr was oriented in the same direction as the pUC lac promoter. A clone in the opposite direction, pRW23D, was created in pUC18 using the blr fragment from pRW23C, followed by insertion of the spectinomycin resistance cassette.

The cloned 358 base pairs region restored the wild type phenotype to the hypersusceptible strain bearing blr::TnphoA, regardless of orientation with respect to the lac promoter (Table 1, pRW23C vs pRW23D). The HindIII/PstI fragment of pACYC 184 (carrying the tet promoter and part of the tet gent) was replaced by the HindIII/PstI fragment from pRW23D. The resulting plasmid, pRWA7, had no vector-derived promoter for blr, yet it completely restored resistance to ampicillin. Therefore the 358 base pairs region appeared to contain all the information needed for complementation.

Beta-lactams are generally bactericidal, and indeed the BLR effect occurred at the level of cell death. To kill the same fraction of logarithmically-growing cells required several-fold less drug for the blr::TnphoA strain than for the wild type, the difference being obviated by cloned blr. The decline in the optical density (probably reflecting lysis) of growing cultures caused by ampicillin also required a lower drug concentration for the insertion mutant.

To determine if the blr locus specified a protein, and if so, where translation might initiate, four different amber mutations (named as though located in the hypothetical ORF6 1) were made within the potential protein coding regions of blr in pRW23C. Amber mutations L24 and L39 (FIG. 1) each prevented the plasmid from restoring the wild type level of ampicillin resistance to mutant RW583 (Table 2A, last 2 rows), indicating that a protein might be involved. Two other amber mutations, Q13 and V20, had no effect (Table 2A), suggesting that the true protein initiated downstream of V20 but upstream of L24 (FIG. 1). Finally, activity was restored in L24 and L39 by the amber suppressor leuX, which inserts the wild type leucine at the amber TAG codon (Table 2B, host S26su). This suppression proved that the active species was a protein. The BLR protein starts with ATG at M21, i.e., at nucleotide 5272, and has 41 residues (FIG. 1). Interestingly, only fifteen other proteins containing ≦50 amino acids have been counted among the more than 4200 proteins of E. coli (Rudd, K. E. Electrophoresis 19, 536-544 (1998)), reflecting in part the difficulty of reliably predicting small proteins from DNA sequences. The only program that predicted BLR (as well as its extensions OFR45 and ORF51) was GeneMark.hmm (Lukashin, A. V. & Borodovsky, M. GeneMark.hmm:new solutions for gene finding Nucl. Acids Res. 26, 1107-1115 (1998)). Genes encoding small proteins in “intergenic” regions have also been recently discovered in yeast (Olivas, W. M., Muhlrad, D. & Parker, R. Analysis of the yeast genome: identification of new non-coding and small OFR-containing RNAs) and in Bacillus subtilis (Bagyan, I., Setlow, B. & Setlow, P. New small, acid-soluble proteins unique to spores of Bacillus subtilis: identification of the coding genes and regulation and function of two of these genes. J.Bacteriol. 180, 6704-6712 (1998).

The BLR protein has a calculated mass of 4556 daltons, an isoelectric point of 6.0 and has all amino acids except cysteine and phenylalanine. A TBLASTN search (comparing the BLR amino acid sequence to in-all-reading-frames translations of the nucleic database) revealed putative BLR homologues of 41 residues in the incomplete genomic sequence of Salmonella typhimurium (85% identity), S. typhi (82%), and S. paratyphi A (82%), and a homologue of 45 residues in Klebsiella pneumoniae (49%). Searches for protein motifs or regions of identity to proteins of known function were not successful. However, half of more of the fusion protein was found in the membrane fraction of cells, judging by alkaline phosphatase (PhoA) activity and by Western blotting using antiPhoA. There is a putative transmembrane domain in BLR upstream from the point of fusion (FIG. 1). The carboxy terminus of BLR is predicted to be in the periplasm, consistent with the presence of PhoA activity, which requires this location (Manoil, C. & Beckwith, J. A. Science 233, 1403-1408 (1986). The Blr-PhoA fusion protein was purified from RW583 membranes by solubilization in dodecylmaltoside and immunoprecipitation with antiPhoA. Quantitation by SDS-PAGE and electroblotting showed that about 30 fusion protein molecules were recovered per cell. The amino terminus proved to be blocked, preventing amino acid sequencing.

In view of the small size of the functional locus, small transcripts were looked for by Northern analysis. RNA was prepared from logarithmic phase cells of ASS111rif (wild type) and RW583 (insertion mutant) by a hot acidic phenol method similar to that described (Emory, S. A. & Belasco, J. F. J. Bacteriol. 172, 4472-4481 (1990)), except that the phenol used was buffered at pH 4.3 with citrate (sigma P4682) and there was no DNase I treatment. The TnphoA insertion caused two blr-hybridizing wild type bands (˜1 and ˜kb) to disappear and two new bands (˜2.9 and ˜1.5 kb) hybridizing with both blr and phoA to appea. The new bands presumably represent two blr-phoA transcripts, to which phoA sequences contribute a calculated 1.4 kb. Since phoA itself has a transcriptional terminator, blr might have two promoters, ˜0.1 kb and ˜1-1.5 kb upstream from the TnphoA junction. The closer one may be that used in the 358 base pairs cloned fragment.

To find the transcriptional start site(s) of the blr locus, a 5′ Rapid Amplification of cDNA Ends (RACE) method was used (Frohman, M. A. Methods Enzymol. 218: 340-356 (1993)) (5′ RACE kit from gibcoBRL/Life Technologies, with concept PCR purification system replacing GlassMax) on RNA prepared from strain ASS111rif as described above. cDNA was made by extending the blr reverse primer R2 (FIG. 1) and tailed it at the 3′ end with dCTPs. A single 210 base pairs PCR product was made using a forward primer that hybridized with the polyC tail and had a 5′ SalI restriction site, together with reverse internal blr primer R3A, having a 5′HindIII site (FIG. 1). The purified product was digested with SalI/HindIII and cloned into pUC19. Of the thousands of transformants, the plasmids from three presumably independent ones were sequenced in the blr sequences), at nucleotide 5233 (FIG. 1). The RACE experiments did not detect a cDNA product long enough to correspond to the larger of the two mRNA species seen in the Northern blots. The program NNPP (Reese, M. G., Harris, N. L., and Eeckrnan, F. H. in Biocomputing: Proceedings of the 1996 Pacific Symposium (ed. Hunter. L. and Klein, T. E.) (World Scientific Publishing Co., Singapore, 1996)) predicted a borderline (score 0.81) prokaryotic promoter for blr, with an upstream limit at nucleotide 5182 and a transcriptional start at nucleotide 5228+/−3 nucleotide. Since this location is close to the experimentally determined start at nucleotide 5233, it is likely a promoter for blr.

The antibiotic that affects peptidoglycan synthesis are involved in murein metabolism. For example, beta-lactam antibiotics react with inner membrane penicillin-binding polypeptides and inhibit their peptide crosslinking activity within the peptidoglycan sacculus in the periplasm. The consequent imbalance in cell wall synthesis/degradation results in cell death in a manner not completely understood (Holtje, J. V. Arch. Microbiol. 164, 243-254 (1995)). One mechanism of BLR action might be to increase a beta-lactamase activity in the cell. It is unlikely that this would be the chromosomally encoded beta-lactamase AmpC since that enzyme does not produce imipenem resistance (Jacoby, G. A. & Sutton, L. Antimicrob. Agents Chemother. 28, 703-705 (1985)) while BLR does (Table 1). Moreover, extracts from cells with the insertion at.blr showed no decrease in beta-lactamase activity by a colorimetric assay using nitrocefin (O'Callaghan, C. A., et al. Antimicrob.Agents Chemother. 1, 283-288 (1972)) or by a bioassay. Therefore increase intrinsic beta-lactamase activity is not a likely explanation for BLR action.

BLR might be part of an uncharacterized membrane-bound efflux pump relatively specific for beta-lactams. Such a pump would have to be capable, like the multidrug efflux system AcrAB (Zgurskaya, H. I. & Nikaido, H. Proc. Natl. Acad. Sci., USA, 96, 7190-7195 (1999)), of expelling beta-lactams from the periplasm. A precedent for involvement of a small, single transmembrane-domain protein in transport is KdpF, a 29 amino acid E. coli protein which is a part, although not an essential one, of a potassium (uptake) transporter complex (Gassel, M., et al. Biochim. Biophys. Acta 1415, 77-84 (1998)). Since cloned blr had no extra effect in a wild type strain (Table 2A, row 1 vs. row 2), any putative efflux complexes in the wild type may already have been fully titrated with chromosomally-encoded BLR molecules.

On the other hand, although preliminary experiments using radiolabelled penicillin showed no differences in amounts or mobilities of penicillin binding polypeptides between the wild type and insertion mutant, it could be that BLR decreases the sensitivity of penicillin-binding polypeptide(s) to beta-lactams or alters a post-binding event (Rodionov, D. G. & Ishiguro, E. E. Antimicrob. Agents Chemother. 40, 899-903 (1996)) which leads to cell death.

TABLE 1

| | Ampi- | Piperi- | Cefe- | Ceftri- | Cefo- | |
|---|---|---|---|---|---|---|
| Strain | cillin | cillin | pime | axone | xitin | Imipene |
| A. | | | | | | |
| ASS111rif | 2 | 1.0 | 0.023 | <0.016 | 2 | 0.38 |
| ASS111rif | 0.5 | 0.25 | <0.016 | <0.016 | 1.0 | 0.19 |
| blr::TnphoA | | | | | | |
| (=RW583) | | | | | | |
| AW1045 | 3 | 1.0 | 0.023 | 0.032 | 3 | 0.38 |
| AW1045 | 0.75 | 0.25 | <0.016 | <0.016 | 1.5 | 0.125 |
| blr::TnphoA | | | | | | |
| WY100 | 3 | 1.5 | 0.047 | 0.125 | 3 | 0.25 |
| WY100 | 0.75 | 0.75 | <0.016 | 0.32 | 1.5 | 0.125 |
| blr::TnphoA | | | | | | |
| B. | | | | | | |
| RW583 + | 0.25 | 1.25 | <0.016 | <0.016 | 0.75 | 0.125 |
| pUC19Sp^{R} | | | | | | |
| (vector) | | | | | | |
| RW583 + | 2 | 1.0 | 0.023 | 0.016 | 1.5 | 0.19 |
| pRW23C | | | | | | |
| (blr⁺, 358 | | | | | | |
| base pairs) | | | | | | |
| RW58s + | 2 | 1.0 | 0.023 | 0.016 | 1.5 | 0.25 |
| pRW23D | | | | | | |
| (blr⁺, 358 | | | | | | |
| base pairs) | | | | | | |
| RW583 + | 0.5 | 0.25 | <0.016 | <0.016 | 0.75 | 0.19 |
| pACYC184 | | | | | | |
| (vector) | | | | | | |
| RW583 + | | | | | | |
| pRW23A | 3 | 1.5 | 0.032 | 0.023 | 2 | 0.38 |
| (blr⁺, 680 base | | | | | | |
| pairs) | | | | | | |

Effect of the blr::TnphoA insertion, and of plasmid clones bearing blr⁺, on susceptibility to B lactams and other antibiotics.
Cells were grown at 37° C. in LB broth supplemented with appropriate antibiotics to mid-log phase, diluted in broth to about 1 × 10⁸ cfu per ml, and the cell suspension was swabbed onto LB agar plates to which E-test strips (AB BIODISK, Piscataway, NJ) were then applied. Strain ASS111rif was derived from ASS111 (recA ΔphoA Δmar) by selection upon rifampicin. AW1045 is the mar⁺ recA⁺ parental strain of ASS111. WY100 is an unrelated strain.

TABLE 2
A. Complementation in RW583

| | | | MIC for |
|---|---|---|---|
| | | | ampi- |
| | | | cillin |
| Host | Host (including | Plasmid | (μg |
| strain | genotype at blr locus) | (insert) | ml⁻¹) |
| ASS11rif | blr⁺ | None | 1.1 |
| ASS11rif | blr⁺ | pRW23C (wild type blr) | 1.0 |
| RW583 | ASS111rifblr::TnphoA | None | 0.13 |
| RW583 | ASS111rifblr::TnphoA | pUC19-Sp^{R} (none) | 0.09 |
| RW583 | ASS111rifblr::TnphoA | pRW23C (wild type blr) | 1.1 |
| RW583 | ASS111rifblr::TnphoA | PQ13tag1 (amber Q13 blr) | 1.1 |
| RW583 | ASS111rifblr::TnphoA | PV20tag1 (amber V20 blr) | 1.2 |
| RW583 | ASS111rifblr::TnphoA | pL24tag1 (amber L24 blr) | 0.13 |
| RW583 | ASS111rifblr::TnphoA | pL39tag3 (amber L39 blr) | ND |

TABLE 3
B. Effect of Leux amber suppressor upon
complementation by inactive plasmids

| | | MIC for ampicillin (μg ml | |
|---|---|---|---|
| | | In non- | |
| Host genotype | Plasmid | suppressor | In leux |
| at blr locus | (insert) | S26 host | amber su |
| blr⁺ | None | 3.3 | 3.5 |
| blr::TnphoA | None | 0.5 | 1.0 |
| blr::TnphoA | pUC19-Sp^{R} (none) | 0.5 | 1.1 |
| blr::TnphoA | pRW23C (wild type blr) | 2.8 | 3.0 |
| Blr::TnphoA | pL24tag1 (amber blr) | 0.9 | 2.1 |
| Blr::TnphoA | pL39tag3 (amber L39 blr) | 0.2 | 0.2 |

Effect of amber mutations in the blr locus of pRW23C in non-suppressor and an amber suppressor and an amber suppressor stain.
The blr locus was mutated in vitro by the “unique restriction site elimination” method. The only SphI site in pRW23C was in the polylinker of the spectinomycin cassette, since the SphI site of pUC19 had been eliminated during cloning of bir.
Amber mutations were verified by sequencing. Strains S26 and its isogenic leuX amber suppressor strain S26su⁺ (from Coli Geentic Stock Center) were made blr::TnphoA by P1 transduction and the transductants were transformed with pUC19-Sp^{R}, pRW23C, pL24tag1, or pL39tag3.

Equivalents

Those skilled in the art will recognize, or be able to ascertain using no more than routine experimentation, numerous equivalents to the specific polypeptides, nucleic acid molecules, methods, assays and reagents described herein. Such equivalents are considered to be within the scope of this invention and are covered by the following claims.

What is claimed is:

1. An isolated nucleic acid molecule comprising the nucleotide sequence set forth in SEQ ID NO:1.

2. An isolated nucleic acid molecule encoding a polypeptide comprising the amino acid sequence set forth in SEQ ID NO:2.

3. An isolated nucleic acid molecule comprising a nucleotide sequence which is at least 50% identical to the nucleotide sequence of SEQ ID NO:1 or a complement thereof selected from the group consisting of;
a) a nucleic acid molecule comprising a fragment of at least 100 nucleotides of a nucleic acid molecule comprising the nucleotide sequence of SEQ ID NO:1 complement thereof,
b) a nucleic acid molecule which encodes a polypeptide comprising an amino acid sequence at least about 50% homologous to the amino acid sequence of SEQ ID NO:2; and
c) a nucleic acid molecule which encodes a fragment of a polypeptide comprising the amino acid sequence of SEQ ID NO:2, wherein the fragment comprises at least 15 contiguous amino acid residues of the amino acid sequence of SEQ ID NO:2.

4. An isolated nucleic acid molecule which hybridizes to the nucleic acid molecule of any one of claims 1, 2, or 3 under stringent conditions.

5. An isolated nucleic acid molecule comprising a nucleotide sequence which is complementary to the nucleotide sequence of the nucleic acid molecule of any one of claims 1, 2, or 3.

6. An isolated nucleic acid molecule comprising the nucleic acid molecule of any one of claims 1, 2, or 3, and a nucleotide sequence encoding a heterologous polypeptide.

7. A vector comprising the nucleic acid molecule of any one of claims 1, 2, or 3.

8. The vector of claim 7, which is an expression vector.

9. A host cell transfected with the expression vector of claim 8.

10. A method of producing a polypeptide comprising culturing the host cell of claim 9 in an appropriate culture medium to, thereby, produce the polypeptide.

11. An isolated polypeptide selected from the group consisting of:
a) a fragment of a polypeptide comprising the amino acid sequence of SEQ ID NO:2, wherein the fragment comprises at least 15 contiguous amino acids of SEQ ID NO:2;
b) a naturally occurring homolog of a BLR polypeptide comprising the amino acid sequence of SEQ ID NO:2, wherein the naturally occurring homolog is isolated from a pathogenic bacterium and is encoded by a nucleic acid molecule which hybridizes to a nucleic acid molecule consisting of SEQ ID NO: 1;
c) a polypeptide which is encoded by a nucleic acid molecule comprising a nucleotide sequence which is at least 50% identical to a nucleic acid molecule comprising the nucleotide sequence of SEQ ID NO:1;
d) a polypeptide comprising an amino acid sequence which is at least 50% identical to the amino acid sequence of SEQ ID NO:2.

12. The isolated polypeptide of claim 11 comprising the amino acid sequence of SEQ ID NO:2.

13. An antibody which selectively binds to a polypeptide of claim 12.

14. An agonist of a BLR polypeptide.

15. An antagonist of a BLR polypeptide.

16. A method for identifying compounds that modulate antibiotic resistance in a microbe comprising:
contacting a BLR polypeptide with a test compound;
determining the ability of the test compound to modulate the activity or expression of a BLR polypeptide; and
selecting those compounds that modulate the activity of the BLR polypeptide to thereby identify compounds that modulate antibiotic resistance.

17. The method of claim 16, wherein the BLR polypeptide is present in a microbial cell.

18. The method of claim 16, wherein the BLR polypeptide is heterologous to the cell in which it is present.

19. The method of claim 17, wherein the microbial cell is an E. coli cell.

20. The method of claim 17, wherein said step of determining comprises measuring the effect of the test compound on the ability of the microbial cell to grow in the presence of an antibiotic.

21. The method of claim 20, wherein the antibiotic is an antibiotic that affects peptidoglycan synthesis selected from the group consisting of a beta lactam, cycloserine, and bacitracin.

22. The method of claim 21, wherein said step of determining comprises measuring the efflux of the test compound or a marker compound from the cell.

23. The method of claim 16, wherein the BLR polypeptide is contacted with the test compound in vitro.

24. A method for identifying compounds that modulate antibiotic resistance in a microbe comprising:
contacting an isolated BLR nucleic acid molecule with a test compound;
determining the ability of the test compound to bind to the isolated BLR nucleic acid molecule; and
selecting those compounds that bind to the BLR nucleic acid molecule to thereby identify compounds that modulate antibiotic resistance.

25. The method of claim 24, wherein the BLR nucleic acid molecule comprises the nucleotide sequence shown in SEQ ID NO:1.

26. A method for identifying a protein that interacts with a BLR nucleotide sequence, comprising:
contacting a BLR nucleotide sequence with a microbial extract under conditions which allow interaction of components of the extract with the BLR nucleotide sequence; and measuring the ability of the BLR nucleotide sequence to interact with the components thereby identify a protein that binds to a BLR nucleotide sequence.

27. A method for identifying a compound that modulates the ability of a BLR nucleic acid molecule to interact with a BLR binding polypeptide, comprising:
contacting at least one of a BLR nucleotide sequence and a BLR binding polypeptide with a test compound under conditions which allow interaction of the compound with at least one of the BLR nucleotide sequence and the BLR binding polypeptide; and measuring the ability of the compound to modulate the interaction of the BLR nucleotide sequence with the BLR binding polypeptide to thereby identify a compound that modulates the ability of a BLR nucleotide sequence to interact with a BLR binding polypeptide.

28. A method for identifying a compound that modulates the ability of a BLR polypeptide to interact with a BLR binding polypeptide, comprising:
contacting at least one of a BLR polypeptide and a BLR binding polypeptide with a test compound under conditions which allow interaction of the compound with at least one of the BLR polypeptide and the BLR binding polypeptide; and measuring the ability of the compound to modulate the interaction of the BLR polypeptide with the BLR binding polypeptide to thereby identify a compound that modulates the ability of a BLR polypeptide to interact with a BLR binding polypeptide.
